(19) United States
(12) Patent Application Publication
(10) Pub. No.: US 2007/0238786 A1
(43) Pub. Date: Oct. 11, 2007

(54) Pharmaceutical Methods, Dosing Regimes And Dosage Forms For The Treatment Of Alzheimer's Disease

(75) Inventors: Adrian Hobden, Salt Lake City, UT (US); Kenton Zavitz, Salt Lake City, UT (US); Gary Mather, Salt Lake City, UT (US); Suzanne Hendrix, Salt Lake City, UT (US)

Correspondence Address: MYRIAD GENETICS INC.;INTELLECUTAL PROPERTY DEPARTMENT, 320 WAKARA WAY, SALT LAKE CITY, UT, 84108, US

(73) Assignee: Myriad Genetics, Incorporated, Salt Lake City, UT (US)

(21) Appl. No.: 11/681,082

(22) Filed: Mar. 1, 2007

Related U.S. Application Data
(63) Continuation of application No. 10/889,971, filed on Jul. 12, 2004.
(60) Provisional application No. 60/486,769, filed on Jul. 11, 2003.
(60) Provisional application No. 60/517,666, filed on Nov. 5, 2003.
(60) Provisional application No. 60/560,685, filed on Apr. 7, 2004.

(51) Int. Cl. A61K 31/192; A61P 25/28

(52) U.S. Cl. 514/568

(57) ABSTRACT

In general, the invention relates to a pharmaceutical dose having R-flurbiprofen as the active ingredient that upon oral administration of a single dose to a fasting subject provides a Cₘₐₓ of about 30-95 μg per mL. When the dose is administered to an individual having mild-to-moderate Alzheimer's disease (or desiring protection against Alzheimer's disease) twice daily for at least 4 months according to the described guidelines, an improvement or lessening in decline of cognitive function as characterized by cognition tests is observed in the patient. The composition of the invention is formulated with one or more pharmaceutically acceptable excipients, salts or carriers.

### CROSS REFERENCE TO RELATED APPLICATIONS

This application is a Continuation of U.S. patent application Ser. No. 10/889,971, filed Jul. 12, 2004; which claims benefit under 35 U.S.C. § 119(e) of application Ser. Nos. 60/486,769, filed Jul. 11, 2003; 60/517,666, filed Nov. 5, 2003; and 60/560,685, filed Apr. 7, 2004, each of which is incorporated herein by reference in its entirety.

### FIELD OF THE INVENTION

The invention provides compositions for the therapeutic or prophylactic treatment of neurodegenerative disorders. The invention provides compositions comprising R-flurbiprofen and one or more pharmaceutically acceptable excipients, diluents, or carriers. The compositions can be used in methods of treating neurodegenerative disorders through the administration of R-flurbiprofen to certain individuals in need of such treatment. The invention further provides methods of improving cognitive function in a variety of Alzheimer's disease patients. The invention has utility for treating and preventing neurodegenerative disorders such as Alzheimer's disease, dementia, and mild cognitive impairment. In addition, the invention encompasses certain doses and dosing regimens for the prevention or treatment of Alzheimer's disease in general, and in particular in certain patient populations.

### BACKGROUND OF THE INVENTION

Dementia is a brain disorder that seriously affects a person's ability to carry out normal daily activities. Among older people, Alzheimer's disease (AD) is the most common form of dementia and involves parts of the brain that control thought, memory, and language. Despite intensive research throughout the world, the causes of AD are still unknown and there is no cure. AD most commonly begins after the age of 60 with the risk increasing with age. Younger people can also get AD, but it is much less common. It is estimated that 3 percent of men and women ages 65 to 74 have AD. Almost half of those ages 85 and older may have the disease. AD is not a normal part of aging. Alzheimer's disease is a complex disease that can be caused by genetic and environmental factors. In the United States alone, four million adults suffer from Alzheimer's disease (AD). Not only does Alzheimer's disease significantly impact the lives of countless families today, it threatens to become even more of a problem as the baby boom generation matures. The economic burden of AD in the United States is estimated to cost over $ 100 billion a year and the average lifetime cost per patient is estimated to be $174,000. Unfortunately, there is no cure available for AD.

In 1906, Dr. Alois Alzheimer, noticed changes in the brain tissue of a woman who had died of an unusual mental illness. In her brain tissue, he found abnormal clumps (now known as amyloid plaques) and tangled bundles of fibers (now known as neurofibrillary tangles) which, today, are considered the pathological hallmarks of AD. Other brain changes in people with AD have been discovered. For example, with AD, there is a loss of nerve cells in areas of the brain that are vital to memory and other mental abilities. Scientists have also found that there are lower levels of chemicals in the brain that carry complex messages back and forth between nerve cells. AD may disrupt normal thinking and memory by blocking these messages between nerve cells.

Plaques and tangles are found in the same brain regions that are affected by neuronal and synaptic loss. Neuronal and synaptic loss is universally recognized as the primary cause in decline of cognitive function. The number of tangles is more highly correlated with the cognitive decline than amyloid load in patients with AD (Albert Proc. Natl. Acad. Sci. U.S.A. 93:13547-13551 (1996)). The cellular, biochemical, and molecular events responsible for neuronal and synaptic loss in AD are not known. A number of studies have demonstrated that amyloid can be directly toxic to neurons (Iversen et al. Biochem. J. 311:1-16 (1995); Weiss et al. J. Neurochem. 62:372-375 (1994); Lorenzo et al. Ann. N.Y. Acad. Sci. 777:89-95 (1996); Storey et al. Neuropathol. Appl. Neurobiol. 2:81-97 (1999), resulting in behavioral impairment. The toxicity of amyloid or tangles is potentially aggravated by activation of the complement cascade (Rogers et al. Proc. Natl. Acad. Sci. U.S.A. 21:10016-10020 (1992); Rozemuller et al. Res. Immunol. 6:646-9 (1992); Rogers et al. Res. Immunol. 6:624-30 (1992); Webster et al. J. Neurochem. 69(1):388-98 (1997)). This suggests involvement of an inflammatory process in AD and neuronal death seen in AD (Fagarasan et al. Brain Res. 723(1-2):231-4. (1996); Kalaria et al. Neurodegeneration 5(4):497-503 (1996); Kalaria et al. Neurobiol Aging. 17(5):687-93 (1996); Farlow Am. J. Health Syst. Pharm. 55 Suppl. 2:S5-10 (1998).

Evidence that amyloid β protein (Aβ) deposition causes some forms of AD was provided by genetic and molecular studies of some familial forms of AD (FAD). (See, e.g., Ii Drugs Aging 7(2):97-109 (1995); Hardy Proc. Natl. Acad. Sci. U.S.A. 94(6):2095-7 (1997); Selkoe J. Biol. Chem. 271(31):18295-8 (1996)). The amyloid plaque buildup in AD patients suggests that abnormal processing of Aβ may be a cause of AD. Aβ is a peptide of 39 to 42 amino acids and forms the core of senile plaques observed in all Alzheimer cases. If abnormal processing is the primary cause of AD, then familial Alzheimer's disease (FAD) mutations that are linked (genetically) to FAD may induce changes that, in one way or another, foster Aβ deposition. There are 3 FAD genes known so far (Hardy et al. Science 282:1075-9 (1998); Ray et al. (1998)). Mutations in these FAD genes can result in increased Aβ deposition.

The first of the 3 FAD genes codes for the Aβ precursor, amyloid precursor protein (APP) (Selkoe J. Biol. Chem. 271(31):18295-8 (1996)). Mutations in the APP gene are very rare, but all of them cause AD with 100% penetrance and result in elevated production of either total Aβ or Aβ₄₂, both in model transfected cells and transgenic animals. The other two FAD genes code for presenilin 1 and 2 (PS1, PS2) (Hardy Proc. Natl. Acad. Sci. U.S.A. 94(6):2095-7 (1997)). The presenilins contain 8 transmembrane domains and several lines of evidence suggest that they are involved in intracellular protein trafficking. Other studies suggest that the presenilins function as proteases. Mutations in the presenilin genes are more common than in the APP gene, and all of them also cause FAD with 100% penetrance. Similar to APP mutants, studies have demonstrated that PS1 and PS2 mutations shift APP metabolism, resulting in elevated Aβ₄₂ production (in vitro and in vivo).

Cyclooxygenases (COX) are major Alzheimer's disease drug targets due to the epidemiological association of NSAID use, whose primary target are cycloxygenases, with a reduced risk of developing Alzheimer's disease (see, e.g., Hoozemans et al. Curr. Drug Targets 4(6):461-8 (2003) and Pasinetti et al. J. Neurosci. Res. 54(1): 1-6 (1998)). The epidemiological studies have indicated that chronic NSAID use appears to reduce the risk of acquiring Alzheimer's disease and/or delay the onset of the disease (see e.g., McGeer et al. Neurology 47(2):425-432 (1996); and Etminan et al. BMJ. 327(7407):128 (2003)). COX-2 selective inhibitors are attractive candidates for long-term drug use since they do not inhibit COX-1 and appear to be less toxic. In support of COX-2 as a target for the treatment for AD, a recent study was published reporting that in mouse models of AD, COX-2 overexpression was related to the neuropathology of AD (Xiang et al. Neurobiol. Aging 23:327-34 (2002)). However, recent clinical trials of specific NSAIDs have called into question the hypothesis the hypothesis that anti-inflammatory drugs are useful for the treatment or prevention of Alzheimer's disease. It was reported that rofecoxib, a COX-2 selective NSAID, at 25 mg daily, failed to show efficacy for treating AD. Naproxen, another NSAID, in the same trial failed to show efficacy in Alzheimer's treatment. See Aisen et al. JAMA 289:2819-26 (2003) and Reines et al. Neurology 62(1):66-71 (2004). These authors concluded that the results with naproxen and rofecoxib do not support the use of NSAIDs for the treatment of AD. Celecoxib, a COX-2—selective NSAID, failed to show efficacy in several recent clinical trials for the treatment of AD. See Jhee et al., “A Double-Blind, Placebo-Controlled Pharmacokinetic (PK), Pharmacodynamic (PD) and Safety Study of Celecoxib Treatment for Four Weeks in Patients with Alzheimer's Disease (AD),” Abstract from 7^{th} International Geneva/Springfield Symposium on Advances in Alzheimer's Therapy (2002); also published in Clinical Research and Regulatory Affairs 21(1): 49-66 (2004)) and Sainati et al. (Abstract from 6^{th} International Stockholm/Springfield Symposium on Advances on Alzheimer's Therapy, Abstract Book 2000; 180). Conversely, it was reported recently that rofecoxib provides neuroprotection in an in vivo Alzheimer's disease excitotoxic model system (Scali et al. Neuroscience 117:909-919 (2003). However, rofecoxib, in a large prevention clinical trial, failed to prevent the development of Alzheimer's disease in patients having mild cognitive impairment. In fact, the results of this trial showed that 6.4% of patients taking rofecoxib developed AD as compared to 4.5% for those taking placebo (see e.g. Visser et al., abstract from Annual meeting of the American College of Neuropsychopharmacology San Juan, Puerto Rico, 2003; and Landers, Wall Street Journal 10 Dec. 2003). Thus, clinical trials have indicated that NSAIDs, as a general class of drugs, are not likely to be useful for treating and/or preventing Alzheimer's disease.

Aβ formation is another target for affecting Alzheimer's disease progression since Aβ amyloid plaques are a central pathological hallmark of the disease. Recently, it was suggested that certain NSAIDs are capable of lowering the level of Aβ₄₂, the form of Aβ associated with plaque formation. United States Patent Application 2002/0128319 to Koo et al., United States Application Publication No. 2002/0128319, discloses the use of an Aβ₄₂ lowering amount of NSAID for treating Alzheimer's disease. R-flurbiprofen, which negligibly inhibits COX activity, was reported in Koo et al. to lower Aβ₄₂ in a transgenic mouse model and CHO cells.

A recent clinical trial using a therapy designed to eliminate Aβ plaques from disease patients failed despite strong evidence of efficacy in animal models (Pfiefer et al. Science 298:1379 (2002)). The Aβ-lowering therapy that worked in animal models caused serious problems in humans. In view of the clinical studies, Atwood et al. (Science 299:1014 (2003)) noted that “[m]ounting evidence indicates that this deposition of amyloid-β may be a neuroprotective response to injury” and “[t]hese results demonstrate yet again the futility of removing a protein, amyloid-β, which has ubiquitous tissue expression, without first understanding its function(s).”

Additionally, gamma-secretase inhibitors, which were designed to alter processing of APP, have turned out to be toxic compounds not likely to be suitable for chronic human use. See De Strooper et al. Nature 398:518-522 (1999); Wong et al. J Biol. Chem. 279:12876-12882 (2004); and Hadland et al. PNAS 98(13):7487-91 (2001). Thus, it is not clear if gamma-secretase inhibitors are a realistic treatment/prevention option. Indeed, as noted recently, mutations in PS-1 associated with AD may cause the disease not through altering Aβ processing, but rather by affecting calcium homeostasis (Mattson, Nature 442:385-386 (2003)).

Several epidemiological studies have reported an association between long-term use of NSAIDs, such as ibuprofen and aspirin, with reduced risk for certain malignancies and neurodegenerative processes characterized by dementia of the Alzheimer's type. A variety of explanations have been given for the reduced cancer and Alzheimer's disease (AD) risk associated with long-term NSAID use. The primary action of NSAIDs appears to be inhibition of cyclooxygenase (COX) activity. Thus, a leading hypothesis is that NSAIDs reduce risk for certain cancers and Alzheimer's disease by affecting the COX enzymes. Other explanations include mediation of apoptosis, modulation of growth factors, and modulation of the nuclear factor kappa B pathway (NF-κB).

U.S. Pat. No. 5,192,753 to Rogers et al. alleges NSAIDs are useful for treating Alzheimer's disease through the inhibition of cyclooxygenase and therefore inhibition of prostaglandin synthesis. U.S. Pat. No. 5,643,960 to Brietner et al. reports the use of COX inhibiting NSAIDs to delay the onset of Alzheimer's symptoms. U.S. Pat. No. 6,025,395 to Brietner et al. relates to the use of COX inhibiting NSAIDs.

Flurbiprofen is a racemic non-steroidal anti-inflammatory drug (NSAID) having a chemical name of (R,S)-(2-fluoro-biphenylyl) propionic acid. 50 milligram (mg) and 100 mg racemic flurbiprofen tablets are marketed as ANSAID® and FROBEN® for the treatment of chronic inflammatory disease.

The literature has described a variety of R-flurbiprofen-containing compositions. Brune et al. J. Clin. Pharmacol. 32:944-952 (1992) discloses the use of tablets containing 50 mg of R-flurbiprofen. Jerussi et al. (J. Clin. Pharmacol. 32:944-952 (1992)) describe the use of 100 mg b.i.d. R-flurbiprofen in investigating gastroduodenal tolerance. Lotsch et al. (Bri. J. Clin. Pharm. 40:339-346 (1995) describe the use 50 mg and 100 mg doses of R-flurbiprofen in pain related chemo-somatosensory evoked potentials in human subjects. The authors concluded that R-flurbiprofen, at these doses, produced an analgesic effect. Geisslinger et al. (Br. J. Clin. Pharmacol. 37(4):392-4 (1994)) discloses the use of 50 mg R-flurbiprofen for examining the disposition of single enantiomers in humans. Oelkers et al. (Br. J. Clin. Pharmacol. 43(2):145-53 (1997)) disclose the use of 75 mg R-flurbiprofen for studying its effects and disposition in blister fluid and human serum. U.S. Pat. No. 5,206,029 to Brune et al. discloses medicaments, containing 10 to 100 mg doses of previously separated flurbiprofen enantiomers, in ratios of from 99.5%:0.5% to 0.5%:99.5%, that are effective for treating pain and inflammatory conditions. U.S. Pat. No. 5,200,198 to Geisslinger et. al. discloses a medicament, containing 10 to 100 mg doses of substantially pure R-flurbiprofen and mixtures containing up to 40% S-enantiomer, that are effective for treating pain and inflammatory conditions.

Of the five drugs currently being used in the US for the treatment of AD, four of them—tacrine (Cognex®), donepezil (Aricept®), rivastigmine (Exelon®), and galantamine (Reminyl®)—are inhibitors of acetylcholinesterase. Another drug, memantine, was recently approved for treating moderate-to-severe AD. More recently it was reported that memantine showed efficacy in treating mild-to-moderate AD. Memantine is a NMDA receptor antagonist.

The drugs currently used for treating AD, including memantine and the acetylcholine esterase inhibitors, are marginally efficacious and have undesirable side-effects. Thus, there is a large unmet need for better and safer drugs.

### SUMMARY OF THE INVENTION

In general, the invention relates to a pharmaceutical composition having R-flurbiprofen as the active ingredient. More specifically, the invention relates to specific dosage formulations or doses (i.e., unit dosage forms) of R-flurbiprofen useful in the treatment or prevention of Alzheimer's disease, e.g., 400 mg, 800 mg, 1200 mg and 1600 mg compositions or daily doses. As described in more detail below, when the dosage for, for example, the 400 mg dosage form, is orally administered in a single dose of the composition of the invention to a fasting subject, it provides a Cₘₐₓ (maximum plasma concentration after administration) of about 30-95 micrograms (μg) per milliliter (mL). When the composition is administered twice daily (b.i.d) for at least 4 months, preferably at least 8 months, and more desirably at least 1 year, it provides an improvement or lessening in decline of cognitive function as characterized by cognition tests, measures of global function, activities of daily living, behavior, biochemical disease marker progression, changes in brain volume, and/or plaque pathology. The cognition tests are those which are capable of measuring cognitive decline in a patient or group of patients. Examples of such tests include cognition tests like the ADAS-cog (Alzheimer's disease Assessment Scale, cognitive subscale) and the MMSE (Mini-Mental State Exam), behavior tests like the NPI (Neuropsychiatric Inventory), daily living activity tests like the ADCS-ADL (Alzheimer's Disease Cooperative Study-Activities of Daily Living), global function test such as the CIBIC-plus (Clinician Interview Based Impression of Change), and CDR sum of boxes (Clinical Dementia Rating). The compositions of the invention are formulated with one or more pharmaceutically acceptable excipients, salts, or carriers. The pharmaceutical compositions of the invention are delivered orally, preferably in a tablet or capsule dosage form. The R-flurbiprofen compositions of the invention can be used in methods for treating, preventing, and prophylaxis against neurodegenerative disorders such as Alzheimer's disease.

In a first aspect, the invention provides a dosage comprising R-flurbiprofen in an amount of about 400 mg to about 800 mg per dose. Oral administration of a single dose to a fasting subject, provides a Cₘₐₓ of about 30-95 μg per mL. Oral administration of the composition of this aspect of the invention twice daily (b.i.d) for at least 4 months, preferably at least 8 months, and more desirably at least 1 year, provides an improvement or lessening of decline in cognitive function as characterized by cognition tests. It is preferred that the improvement in decline in cognitive function is at least 25% as compared to a control, more preferably at least 40%, and even more desirably at least 60%. The control may be a plurality of individuals treated with placebo, or may be the expected decline in a test of cognition over a period of time. For example, an individual having probable mild-to-moderate Alzheimer's disease, who is treated with placebo, is expected to score approximately 5.5 points higher on the ADAS-cog test after a specified period of time of treatment (e.g., 1 year) whereas an individual treated with a composition of the invention for the same period of time will score only about 2.2 points higher on the ADAS-cog scale, e.g., will show about a 60% improvement in decline; or only about 3.3 points higher, e.g., will show about a 40% improvement in decline in cognitive function. Of course, the actual numeric score will depend upon the test given. For example, a higher number on the MMSE indicates better cognition, and a lower score (i.e., below 26) indicates some degree of dementia.

Desirably, the oral dose is provided in capsule or tablet form. In a specific embodiment of this aspect of the invention, the dosage is provided as a pharmaceutical composition composed of R-flurbiprofen, a pharmaceutically acceptable salt, a release agent, and optionally additional ingredients. In another specific embodiment of this aspect of the invention, the dosage is provided as a pharmaceutical composition in a unit dosage form that is a tablet composed of R-flurbiprofen, microcrystalline cellulose, colloidal silicon dioxide, and magnesium stearate. In another specific embodiment of this aspect of the invention, the dosage is provided as a pharmaceutical composition in a unit dosage form that is a coated tablet composed of R-flurbiprofen, microcrystalline cellulose, colloidal silicon dioxide, and magnesium stearate, all coated in a mixture of lactose monohydrate, hydroxyl propyl methyl cellulose, titanium dioxide, tracetin/glycerol triacetate, and iron oxide. In another specific embodiment of this aspect of the invention, the dosage is provided as a pharmaceutical composition in a unit dosage form that is a capsule composed of R-flurbiprofen, microcrystalline cellulose, colloidal silicon dioxide, and magnesium stearate.

In a related aspect, the invention provides for a method of treating an individual having, or suspected of having, Alzheimer's disease, comprising administering R-flurbiprofen, wherein said administration provides a Cₘₐₓ of about 30 to about 95 μg per mL. In a more specific embodiment, said Cₘₐₓ is between 40 and 80 μg per mL. The invention further provides a method of improving a decline in a measure of cognitive function of an individual comprising administering R-flurbiprofen to said individual, wherein said administering results in the reduction of the decline in said measure of cognitive function as compared to a control. In a specific embodiment, said control is the decline in said measure of cognitive function in an individual not given R-flurbiprofen, wherein said individual has or is suspected of having Alzheimer's disease. In another specific embodiment, said control is the average decline in said measure of cognitive function in a plurality of individuals not given flurbiprofen, wherein said individuals have or are suspected of having Alzheimer's disease. In another specific embodiment, said reduction in said decline in said measure of cognitive function is at least 25% compared to said control. In another specific embodiment, said reduction in said decline in said measure of cognitive function is at least 40% compared to said control. In another specific embodiment, said improvement in said decline in said measure of cognitive function is at least 60% compared to said control. In another specific embodiment, said measure of cognitive function is an ADAS-cog test. In a more specific embodiment, said reduction in decline is about 2.2 points in the ADAS-cog test over one year. In another more specific embodiment, said reduction in decline is about 3.3 points in the ADAS-cog test over one year. In another specific embodiment, said R-flurbiprofen is administered in a dose of about 400 mg twice daily. In another specific embodiment, said R-flurbiprofen is administered in a dose of about 800 mg twice daily.

In a second aspect, the invention provides a dosage having R-flurbiprofen in an amount of about 400 mg to about 800 mg per dose that is suitable for providing an improvement or lessening of decline in biochemical disease marker progression. Oral administration of a single dose to a fasting subject provides a Cₘₐₓ of about 30-95 μg per mL. Oral administration of the composition of this aspect of the invention twice daily (b.i.d) for at least 4 months, preferably at least 8 months, and more desirably at least 1 year, provides an improvement or lessening of decline in biochemical disease marker progression. Examples of biochemical disease markers include, for example, amyloid beta peptide (Aβ), Aβ₄₂, and tau. It is preferred that the lessening in decline in biochemical disease marker progression is at least 10% as compared to individuals treated with placebo, more preferably at least 20%, and more desirably at least 40%. Desirably, the oral dose is provided in capsule or tablet form. In a specific embodiment of this aspect of the invention, the dosage is provided as a pharmaceutical composition that is composed of R-flurbiprofen, a pharmaceutically acceptable salt, a release agent, and optionally additional ingredients. In another specific embodiment of this aspect of the invention, the dosage is provided as a pharmaceutical composition in a unit dosage form that is a tablet composed of R-flurbiprofen, microcrystalline cellulose, colloidal silicon dioxide, and magnesium stearate. In another specific embodiment of this aspect of the invention, the dosage is provided as a pharmaceutical composition in a unit dosage form that is a coated tablet composed of R-flurbiprofen, microcrystalline cellulose, colloidal silicon dioxide, and magnesium stearate, all coated in a mixture of lactose monohydrate, hydroxyl propyl methyl cellulose, titanium dioxide, tracetin/glycerol triacetate, and iron oxide. In another specific embodiment of this aspect of the invention, the dosage is provided as a pharmaceutical composition in a unit dosage form that is a capsule composed of R-flurbiprofen, microcrystalline cellulose, colloidal silicon dioxide, and magnesium stearate.

In a related aspect, the invention provides for a method of improving or lessening the rate of decline in (i.e., reversing or slowing the progression of), Alzheimer's disease in an individual having, or suspected of having, Alzheimer's disease, comprising administering R-flurbiprofen to said individual. Disease progression may be monitored by one or more Alzheimer's disease markers. In a specific embodiment, said administration provides a Cₘₐₓ of about 30 to about 95 μg per mL. In a more specific embodiment, said Cₘₐₓ is between 40 and 80 μg per mL. In a specific embodiment, said administration is continued at least once a day for at least four months. In another specific embodiment, said administration is continued at least once a day for at least eight months or for at least twelve months. In a specific embodiment, said disease marker is amyloid beta peptide (Aβ), Aβ₄₂, or tau. In another specific embodiment, said R-flurbiprofen is administered in a dose of about 400 mg twice daily. In another specific embodiment, said R-flurbiprofen is administered in a dose of about 800 mg twice daily.

In a third aspect, the invention provides a dosage having R-flurbiprofen in an amount of about 400 mg to about 800 mg per dose that is suitable for providing an improvement or lessening of decline in plaque pathology associated with AD. Oral administration of a single dose to a fasting subject, provides a Cₘₐₓ of about 30-95 μg per mL. Oral administration of the composition of this embodiment twice daily for at least 4 months, preferably at least 8 months, and more desirably at least 1 year, provides an improvement or lessening of decline in plaque pathology. It is preferred that the lessening in, i.e., improvement in, decline in plaque pathology is at least 10% as compared to individuals treated with placebo, preferably at least 20%, and even more desirably at least 40%. Plaque pathology can be assessed by a variety of techniques including, for example, positron emission tomography. Desirably, the oral dose is provided in capsule or tablet form. In a specific embodiment of this aspect of the invention, the dosage is a provided as a pharmaceutical composition composed of R-flurbiprofen, a pharmaceutically acceptable salt, a release agent, and optionally additional ingredients. In another specific embodiment of this aspect of the invention, the dosage is provided as a pharmaceutical composition in a unit dosage form that is a tablet composed of R-flurbiprofen, microcrystalline cellulose, colloidal silicon dioxide, and magnesium stearate. In another specific embodiment of this aspect of the invention, the dosage is provided as a pharmaceutical composition in a unit dosage form that is a coated tablet composed of R-flurbiprofen, microcrystalline cellulose, colloidal silicon dioxide, and magnesium stearate all coated with a mixture of lactose monohydrate, hydroxyl propyl methyl cellulose, titanium dioxide, tracetin/glycerol triacetate and iron oxide.

In a related aspect, the invention provides for a method of improving, or lessening a decline in, plaque pathology associated with Alzheimer's disease in an individual having, or suspected of having, Alzheimer's disease, comprising administering R-flurbiprofen to said individual. In a specific embodiment, said administration provides a Cₘₐₓ of about 30 to about 95 μg per mL. In a more specific embodiment, said Cₘₐₓ is between 40 and 80 μg per mL. In a specific embodiment, said administration is continued at least once a day for at least four months. In another specific embodiment, said administration is continued at least once a day for at least eight months or for at least twelve months. In another specific embodiment, said R-flurbiprofen is administered in a dose of about 400 mg twice daily. In another specific embodiment, said R-flurbiprofen is administered in a dose of about 800 mg twice daily.

In a fourth aspect, the invention provides a method of treating Alzheimer's disease comprising administering to a patient in need of such treatment, a pharmaceutical composition comprising an effective amount of R-flurbiprofen and one or more pharmaceutically acceptable excipients, salts, or carriers. A dose of an effective amount, upon oral administration to a fasting subject, provides a Cₘₐₓ of about 30-95 μg per mL. Oral administration of the composition of this aspect of the invention twice daily for at least 4 months, preferably at least 8 months, and more desirably at least 1 year provides an improvement or lessening in decline of cognitive function as characterized by cognition tests, biochemical disease marker progression, and/or plaque pathology. Desirably, the oral dose is provided in capsule or tablet form. According to this aspect of the invention, a patient in need of treatment is administered an Alzheimer's disease treating effective amount of a pharmaceutical composition having R-flurbiprofen and one or more pharmaceutically acceptable salts, excipients and carriers. The method of this aspect of the invention involves identifying individuals likely to have mild-to-moderate Alzheimer's disease. Individuals having probable mild-to-moderate Alzheimer's disease can be diagnosed by any method available to the ordinary artisan skilled is such diagnoses. For example, diagnosis can be according to DSM-IV (TR) and/or meets NINCDS-ADRDA criteria for probable AD. According to this aspect of the invention, individuals with probable mild-to-moderate AD take an oral dose of a pharmaceutical composition, twice-a-day (e.g., two tablets containing 400 mg of R-flurbiprofen twice daily, or one tablet containing 400 mg of R-flurbiprofen twice daily) for at least 90 days, preferably 120 days, more preferably at least 180 days and even more desirably at least 365 days. Individuals undergoing such treatment are likely to see an improvement or lessening in decline of cognitive function, an improvement or lessening in decline in biochemical disease marker progression, and/or an improvement or lessening in decline in plaque pathology. A lessening in decline in cognitive function can be assessed using test of cognitive function like the ADAS-cog. For example, an individual treated with placebo having probable mild-to-moderate Alzheimer's disease is expected to score approximately 5.5 points higher on the ADAS-cog test after a specified period of time of treatment (e.g., 1 year) whereas an individual treated with the composition of this aspect of the invention for the same period of time will score approximately 2.2 points higher on the ADAS-cog scale, i.e., a 60% decrease in decline, or 3.3 points higher, i.e., a 40% decrease in decline in cognitive function.

In a fifth aspect, the invention provides a method of preventing the onset of Alzheimer's disease comprising administering to a patient in need of or desiring such treatment, a pharmaceutical composition comprising an effective amount of R-flurbiprofen and one or more pharmaceutically acceptable excipients. A dose of an effective amount upon oral administration to a fasting subject provides a Cₘₐₓ of about 30-95 μg per mL. Oral administration of the R-flurbiprofen composition twice daily for at least 4 months, preferably at least 8 months, and more desirably at least 1 year, delays the onset of decline of cognitive function, biochemical disease marker progression, and/or plaque pathology. According to this embodiment, an individual desiring or needing preventative treatment against the onset of AD is administered twice daily a dose having from about 400 mg to about 800 mg of R-flurbiprofen. Desirably, the oral dose is provided in capsule or tablet form. The preventive treatment is preferably maintained as long as the individual continues to desire or need the treatment. Individuals needing or desiring preventative treatment against AD can be those having risk factors for developing AD. For example, risk factors for developing AD can be genetic factors or environmental factors. In one embodiment, the risk factor is age. Genetic risk factors can be assessed in a variety of ways, such as ascertaining the family medical history of the individual, or performing a genetic test to identify genes that confer a predisposition for developing AD. Additionally, risk factors can be assessed by monitoring genetic and biochemical markers.

In a sixth aspect, the invention provides a method of decelerating the onset of Alzheimer's disease comprising administering to a patient in need of such treatment, a pharmaceutical composition comprising an effective amount of R-flurbiprofen and one or more pharmaceutically acceptable excipients, wherein a dose of an effective amount upon oral administration to a fasting subject provides a Cₘₐₓ of about 30-95 μg per mL. Oral administration of the R-flurbiprofen composition twice daily for at least 4 months, preferably 8 months, and more desirably 1 year provides a deceleration in decline of cognitive function, biochemical disease marker progression, and/or plaque pathology. According to this aspect of the invention, an individual having mild cognitive impairment that is likely progress to AD is identified. Alternatively, the individual can be in the prodromal stage of AD development. Upon identification of an individual having mild cognitive impairment likely to progress to Alzheimer's disease or being in the prodromal stage of AD development, a preventive treatment regimen is prescribed for the patient. The preventive treatment regimen involves administering to the individual in need or desiring such treatment a pharmaceutical composition sufficient to decelerate the onset of Alzheimer's disease. The R-flurbiprofen composition for use in this aspect of the invention is designed in such as to be suitable for chronic long-term use with a prophylactic effect.

In a seventh aspect, the invention provides a method of selecting a regimen for treating cognitive decline in an individual desiring such treatment. The method of this aspect involves evaluating risk factors for cognitive decline. Evaluation of risk factors can include genetic testing for predisposing genes, alleles, and polymorphisms. Risk factors also refer to environmental factors like stroke, brain injury, age, and diet. Depending on the risk factor or factors associated with a particular patient a particular treatment regimen is selected for treating cognitive decline. For example, mutations in a Familial Alzheimer's disease genes such as APP, PS1 or PS2, are a risk factor. Another risk factor for cognitive decline is age. Head trauma is another risk factor for cognitive decline. Based on the patient's risk factors, a physician will prescribe a particular therapeutic treatment or prophylactic treatment suitable for the patient.

In an eighth aspect, the invention relates to a method for improving cognitive function. More particularly, this aspect of the invention provides a method for improving cognitive function in individuals experiencing cognitive decline such as that experienced by Alzheimer's disease patients. The invention is based on the discovery that Alzheimer's disease patients that have experienced cognitive decline as a result of the disease can experience an improvement in cognition when administered a cognition improving effective amount of a pharmaceutical composition having R-flurbiprofen as the active ingredient. In one embodiment, the invention provides a method for improving cognitive function in individuals experiencing cognitive decline. According to this method, an individual in need of or desiring treatment (e.g., a patient having Alzheimer's disease or mild cognitive impairment), is administered a composition having R-flurbiprofen in an amount of about 100 mg to about 1800 mg per day for at least 4 weeks, preferably at least 4 months, and more preferably at least 6 months. The composition used in the invention is formulated with one or more pharmaceutically acceptable excipients, salts, or carriers. The pharmaceutical composition can be delivered orally, preferably in a tablet or capsule dosage form. Oral administration of a single dose of the cognition improving effective amount of R-flurbiprofen to a fasting subject provides a Cₘₐₓ of about 30-95 μg per mL. Oral administration of the R-flurbiprofen composition twice daily (b.i.d) for at least 4 weeks, preferably at least 4 months, even more preferably at least 6 months, and more desirably at least 1 year, provides an improvement in cognitive function as characterized by cognition tests. It is preferred that the improvement in cognitive function is statistically significant as compared to individuals treated with placebo. For example, where the ADAS-cog test is used as a cognition test, an individual treated with placebo having probable mild-to-moderate Alzheimer's disease is expected to score approximately 5.5 points higher on the ADAS-cog test after a specified period of time of treatment (e.g., 1 year) whereas an individual (having mild to moderate Alzheimer's disease) treated with the R-flurbiprofen composition for the same period of time will score no higher on the ADAS-cog scale or will have a better, i.e., lower, score. Desirably, the oral dose is provided in capsule or tablet form. In a specific embodiment of this aspect of the invention, the dosage is provided as a pharmaceutical composition composed of R-flurbiprofen, a pharmaceutically acceptable salt, a release agent, and optionally additional ingredients.

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### BRIEF DESCRIPTION OF THE DRAWINGS

FIG. 1 depicts a One Compartment Pharmacokinetic Model used in a pharmacokinetic study as described in Example 5.

FIG. 2 depicts pharmacokinetic results obtained from the study disclosed in Example 5. Graph presents the mean concentration of a 200 b.i.d., 400 b.i.d., or 800 b.i.d. dose in the plasma of individuals from 0 to 25 hours after administration. Circles: actual mean plasma concentrations for each dosage group. Line: predicted plasma concentration for each dosage group using the model of FIG. 1.

### DETAILED DESCRIPTION OF THE INVENTION

In general, the invention relates to a pharmaceutical composition having R-flurbiprofen as the active ingredient. The invention encompasses oral compositions that, upon administration of a dose of said pharmaceutical composition to a subject, provides pharmacokinetic and therapeutic characteristics particularly useful in the methods of the invention. The invention also encompasses the use of the inventive composition according to the treatment regimens of the invention by an individual desiring or needing such treatment, thus providing an improvement or lessening in decline of cognitive function, biochemical disease marker progression, and/or plaque pathology associated with neurodegenerative disorders such as AD. The composition of the invention is formulated with one or more pharmaceutically acceptable excipients, salts, or carriers. The pharmaceutical composition of the invention is delivered orally, preferably in a tablet or capsule dosage form. The R-flurbiprofen composition of the invention can be used in methods for treating, preventing, and prophylaxis against neurodegenerative disorders such as Alzheimer's disease.

### 1.1 Definitions

As used herein, the term “preventing an increase in a symptom” refers to both not allowing a symptom to increase or worsen, as well as reducing the rate of increase in the symptom. For example, a symptom can be measured as the amount of particular disease marker, i.e., a protein. Preventing an increase, according to the definition provided herein, means that the amount of the protein does not increase or that the rate at which it increases is reduced.

As used herein, the term “treating Alzheimer's disease” refers to a slowing of or a reversal of the progress of the disease in an individual that has been diagnosed as having, or has one or more indicia of, mild Alzheimer's disease, as diagnosed by a test of cognition. Treating Alzheimer's disease includes reducing, lessening or improving one or more of the symptoms of the disease.

As used herein, the term “preventing Alzheimer's disease” refers to a slowing of, or stopping, the onset of the disease or of one or more of the symptoms thereof. In particular, the term means slowing or stopping the onset of one or more aspects of Alzheimer's disease that would otherwise lead to a diagnosis of at least mild Alzheimer's disease on one or more tests of cognition.

The term “with reduced gastrointestinal toxicity” as used herein means that the administration of R-flurbiprofen is less ulcerogenic to the gastrointestinal tract of the human or other mammal than the corresponding racemate, or S-flurbiprofen. One measure of ulcerogenic activity is the small bowel ulcer score. A rat is treated daily through oral administration of the R-flurbiprofen for 30 days. At the end of the 30 days, the rat is sacrificed and the intestines removed. Lesions of appreciable size in the mucosa are measured. A cumulative score equaling the sum of the diameters of the ulcers measured are reported as the ulcer score. An ulcer score essentially equal to that of a control rat, or a reduction of the ulcer score of at least 50 to 90%, preferably at least 80%, as compared to the corresponding S-enantiomer or NSAID racemate, is considered a reduction in gastrointestinal toxicity. In another embodiment, the term “with reduced gastrointestinal toxicity” refers the ability to administer a lower amount of flurbiprofen such that unwanted gastrointestinal toxicity side-effects are reduced.

As used herein, the term “R-flurbiprofen” refers to the R-enantiomer of the non-steroidal anti-inflammatory drug flurbiprofen. R-flurbiprofen can be administered as a substantially pure R-enantiomer or as part of a racemic mixture. In a preferred embodiment, the amount of R-flurbiprofen is adjusted to avoid adverse effects associated with the S enantiomer of flurbiprofen. The term “substantially free of the (S)-stereoisomer” as used herein means that the composition contains a greater proportion of the R-enantiomer in relation to the S-enantiomer of flurbiprofen. In a preferred embodiment the term “substantially free of its S-stereoisomer” as used herein means that the composition contains at least 90% by weight of R-flurbiprofen and 10% by weight or less of S-flurbiprofen; in a more preferred embodiment at least 95% R-flurbiprofen and 5% by weight or less of its S-enantiomer. These percentages are based on the total amount of flurbiprofen present in the composition. In the certain preferred embodiments the term “substantially free of its S-stereoisomer” means that the composition contains approximately 99% by weight of R-flurbiprofen, and 1% or less of S-flurbiprofen. In another preferred embodiment, the term “substantially free of its S-stereoisomer” as used herein means that the composition contains greater than 99% by weight of the R-enantiomer of flurbiprofen, again based on the total amount of flurbiprofen present. The terms “substantially optically pure R-isomer of flurbiprofen,” “optically pure R-isomer of flurbiprofen,” “optically pure R-flurbiprofen” and “R-isomer of flurbiprofen” are also encompassed by the above-described amounts. The term “substantially free” indicates that the amount of S-flurbiprofen, if any, present in the composition is insufficient to elicit an adverse effect in the patient to whom the composition is administered or, at most elicits an adverse effect that is tolerable to the patient and is outweighed by the beneficial effect or effects.

As used herein, the term “unit dosage form” refers to a physically discrete unit, such as a capsule or tablet suitable as a unitary dosage for a human patient. Each unit contains a predetermined quantity of R-flurbiprofen that was discovered as a result of this invention to produce the desired pharmacokinetic profile which yields the desired therapeutic effect. The dosage unit is composed of R-flurbiprofen in association with at least one pharmaceutically acceptable carrier, salt, excipient, or combination thereof.

As used herein, the term “dose” or “dosage” refers the amount of active ingredient that an individual takes or is administered at one time. For example, an 800 mg R-flurbiprofen dose refers to, in the case of a twice-daily dosage regimen, a situation where the individual takes 800 mg R-flurbiprofen in the morning and 800 mg R-flurbiprofen in the evening. The 800 mg R-flurbiprofen dose can be divided into two or more dosage units, e.g., two 400 mg R-flurbiprofen tablets or two 400 mg R-flurbiprofen capsules.

As used herein, “about” indicates a range of +/−20%. For example, “about 400 mg R-flurbiprofen” means a range of from 320 mg to 480 mg R-flurbiprofen.

As used herein, “decline,” when used to characterize a disease such as Alzheimer's, or a symptom or marker thereof, means a worsening or progression of the disease, symptom or marker thereof over time from less-advanced to more-advanced. In the case of Alzheimer's disease, a decline indicates a worsening or increase in the severity of one or more behavioral, cognitive, biochemical or clinical parameters of the condition. “Decline” also indicates a progression of one or more scores on a cognition test that indicate a worsening of the condition, regardless of whether the actual, raw scores increase or not.

As used herein, “Alzheimer's disease” and “AD” are equivalent.

### 1.2 Patient Population

Any individual having, or suspected of having, a neurodegenerative disorder, such as Alzheimer's disease, may be treated using the compositions and methods of the present invention. Individuals who would particularly benefit from the compositions and methods of the invention include those individuals diagnosed as having mild to moderate Alzheimer's disease according to a medically-accepted diagnosis, such as, for example the NINCDS-ADRDA criteria. Progression of the disease may be followed by medically accepted measure of cognitive function, such as, for example, the Mini-Mental State Exam (MMSE; see Mohs et al. Int. Psychogeriatr. 8:195-203 (1996)); ADAS-Cog (Alzheimer Disease Assessment Scale-Cognitive; see Galasko et al. Alzheimer Dis Assoc Disord, 11 suppl 2:S33-9 (1997)); Behavioral Pathology in Alzheimer's Disease Rating Scale (BEHAVE-AD); Blessed Test; CANTAB—Cambridge Neuropsychological Test Automated Battery; CERAD (The Consortium to Establish a Registry for Alzheimer's Disease) Clinical and Neuropsychological Tests (includes MMSE); Clock Draw Test; Cornell Scale for Depression in Dementia (CSDD); Geriatric Depression Scale (GDS); Neuropsychiatric Inventory (NPI); the 7 Minute Screen; the Alzheimer's Disease Cooperative Study Activities of Daily Living scale (ADCS-ADL; see McKhann et al. Neurology 34:939-944 (1984)); the DSM-IV (Diagnostic and Statistical Manual of Mental Disorders—Fourth Edition (DSM-IV), published by the American Psychiatric Association, Washington D.C., 1994); or the NINCDS-ADRDA criteria (see Folstein et al. J. Psychiatr. Res. 12:189-198 (1975)). Individuals diagnosed as having probable AD can be identified as having a mild-to-moderate form of the disease by an accepted measure of cognitive function such as the MMSE. In addition, methods that allow for evaluating different regions of the brain and estimating plaque and tangle frequencies can be used. These methods are described by Braak et al. Acta Neuropathol 82:239-259 (1991); Khachaturian Arch. Neuro. 42:1097-1105 (1985); Mirra et al. (1991) Neurology 41:479-486; and Mirra et al. Arch Pathol Lab Med 117:132-144 (1993). The severity of AD is generally determined by one of the initial tests provided above. For example, MMSE scores of 26-19 indicate mild AD, while scores from 18-10 indicate moderate AD.

Diagnoses of Alzheimer's disease based on these tests are recorded as presumptive or probable, and may optionally be supported by one or more additional criteria. For example, a diagnosis of Alzheimer's disease may be supported by evidence of a family history of AD; non-specific changes in EEG, such as increased slow-wave activity; evidence of cerebral atrophy on CT with progression documented by serial observation; associated symptoms such as depression, insomnia, incontinence, delusions, illusions, hallucinations, catastrophic verbal, emotional or physical outbursts, sexual disorders, weight loss, and/or attendant neurologic abnormalities, such as increased muscle tone, myoclonus or gait disorder, etc.

Additionally, amyloid deposits, generally associated with AD, may be detected through the use of positron emission tomography (PET) using an amyloid-specific tracer such as Pittsburgh Compound-B (PIB). See Klunk et al., Ann. Neurol. 55(3):306-309 (2004). Increased amyloid deposits in the frontal, parietal, temporal and occipital cortices, and in the striatum, relative to normal brain tissue, as visualized, for example by PIB, support a diagnosis of AD. Generally, a greater number and density of amyloid deposits indicates more advanced AD.

The invention encompasses the treatment of an individual preferably having mild to moderate AD, to the extent that individual has AD, whether or not one or more non-AD neurodegenerative diseases or conditions are previously, concurrently or subsequently diagnosed.

The compounds and methods of the present invention are useful for individuals who have received prior medication for AD, as well as individuals who have received no prior medication for AD, and is useful for individuals currently receiving medication for AD other than R-flurbiprofen, and for individuals not receiving medication for AD other than R-flurbiprofen.

Individuals of any age may be treated by the methods of the invention, with the pharmaceutical compositions of the invention; however, the invention encompasses a preferred embodiment for treating or preventing Alzheimer's disease in individuals between the ages of 55 and 80. In various embodiments, individuals treated by the therapeutic or prophylactic methods of the invention may be from 55 to 70 years of age, 60 to 80 years of age, 55 to 65 years of age, 60 to 75 years of age, 65 to 80 years of age, 55 to 60 years of age, 60 to 65 years of age, 65 to 70 years of age, 70 to 75 years of age, 75 to 80 years of age, or 80 years old and older.

Thus, in one embodiment, the invention provides a method of treating an individual known or suspected of having Alzheimer's disease comprising administering an effective amount of R-flurbiprofen. In a specific embodiment, said individual is diagnosed as having mild to moderate Alzheimer's disease. In a more specific embodiment, said individual is diagnosed by a cognitive test as having mild to moderate AD. In a more specific embodiment, said cognitive test is the Mini-Mental State Exam (MMSE). In an even more specific embodiment, said individual has a score in said MMSE of from 26 to 19, inclusive. In another more specific embodiment, said individual has a score in said MMSE of from 18 to 10, inclusive. In another specific embodiment, said individual has a score in said MMSE of 26 to 10, inclusive.

In other embodiments, the invention provides a method of treating an individual known or suspected of having Alzheimer's disease comprising administering an effective amount of R-flurbiprofen, wherein said individual is concurrently taking a second drug for the treatment of Alzheimer's disease. In a further embodiment, said individual has been diagnosed as having mild to moderate Alzheimer's disease. In a specific embodiment, said second drug is an acetylcholinesterase (AChE) inhibitor. In a more specific embodiment, said AChE inhibitor is Galanthamine (galantamine, Reminyl); E2020 (Donepezil, Aricept); Physostigmine; Tacrine (tetrahydroaminoacridine, THA); Rivastigmine; Phenserine; Metrifonate (Promem); or Huperazine, or a combination of any of the foregoing. In another embodiment, said second drug is a drug other than an acetylcholinesterase inhibitor. In a preferred embodiment, the method or compositions of the invention are used in patients or individuals undergoing therapy with Aricept. The invention also encompasses methods of treating patients refractory to, or who no longer show improvement with, conventional AD therapy.

In another embodiment, said individual is concurrently taking a non-drug substance for the treatment of Alzheimer's disease. In a specific embodiment, said non-drug substance is an anti-oxidant. In a more specific example, said anti-oxidant is vitamin C or vitamin E. In an even more specific embodiment, said vitamin C is taken in a dose of 500-1000 mg per dose of R-flurbiprofen. In another even more specific embodiment, said vitamin E is taken in a dose of 400-800 IU per dose of R-flurbiprofen. In this regard, the invention encompasses the use of one or more such anti-oxidants as an adjunct to therapy for Alzheimer's disease, and not primarily as a nutritional supplement.

In another embodiment, the invention provides a method of treating an individual diagnosed as having mild to moderate Alzheimer's disease comprising administering an effective amount of R-flurbiprofen, wherein said individual has, prior to taking R-flurbiprofen, taken a second drug for the treatment of Alzheimer's disease. In a specific embodiment, said second drug is an acetylcholinesterase (AChE) inhibitor. In a more specific embodiment, said ACE inhibitor is Galanthamine (galantamine, Reminyl); E2020 (Donepezil, Aricept); Physostigmine; Tacrine (tetrahydroaminoacridine, THA); Rivastigmine; Phenserine; Metrifonate (Promem); or Huperazine, or a combination of any of the foregoing. In another embodiment, said second drug is a drug other than an acetylcholinesterase inhibitor.

In another embodiment, said individual has, prior to taking R-flurbiprofen, taken a non-drug substance for the treatment of Alzheimer's disease. In a specific embodiment, said non-drug substance is an anti-oxidant. In a more specific example, said anti-oxidant is vitamin C or vitamin E. In an even more specific embodiment, said vitamin C is taken in a dose of 500-1000 mg per dose. In another even more specific embodiment, said vitamin E is taken in a dose of 400-800 IU per dose. In this regard, the invention encompasses the use of one or more such anti-oxidants as an adjunct to therapy for Alzheimer's disease, and not primarily as a nutritional supplement.

Although any individual having, or suspected of having, Alzheimer's disease may be treated with R-flurbiprofen as described elsewhere herein, certain patient subpopulations may be identified that would especially benefit from the use of R-flurbiprofen. For example, the invention encompasses a preferred method wherein R-flurbiprofen is used in individuals who do not have: (1) a history in the past 2 years of epilepsy, focal brain lesion, head injury with loss of consciousness and/or immediate confusion after the injuries; (2) DSM-IV (TR) criteria for any major psychiatric disorder including psychosis, major depression, bipolar disorder, alcohol or substance abuse; (3) a history of hypersensitivity to flurbiprofen or other NSAIDs including COX-2 specific inhibitors; (4) a history of upper GI bleeding requiring transfusion or surgery within the past 3 years; (5) active gastric or duodenal ulcer disease; (6) a history of NSAID-associated ulcers; (7) active malignancy, or a history of active malignancy, except for basal cell carcinoma or squamous cell carcinoma of the skin; (8) chronic or acute renal, hepatic or metabolic disorder defined by creatinine>1.5 mg/dL, AST>2.5×Upper Limit of Normal (ULN); or ALT>2.5×ULN; uncontrolled cardiac conditions (New York Heart Association Class III or IV); (9) current anticoagulant therapy such as warfarin; or (10) current treatment with any CYP2C9 inhibitor (for example, amiodarone, fluconazole, fluvoxamine, isoniazid, phenylbutazone, probenicid, sulfamethoxazole, sulfaphenazole, trimethoprim, zafirlukast; danshen (Salvia miltiorrhiza); Lycium barbarum) or the CYP2C9 substrates fluvastatin, tolbutamide, or glyburide (glibenclamide); or who do not show chronic use of NSAIDs at any dose or aspirin>325 mg per day.

In yet another embodiment, the invention provides a method of slowing cognitive decline in an individual suspected of having mild cognitive impairment (MCI) comprising administering to the individual an effective amount of R-flurbiprofen. Mild cognitive impairment is a clinical condition between normal aging and Alzheimer's disease characterized by memory loss greater than expected for the particular age of the individual yet the individual does not meet the currently accepted definition for probable Alzheimer's disease. See, e.g., Petersen et al. Arch. Neurol. 58:1985-1992 (2001); Petersen Nature Rev. 2:646-653 (2003); and Morris et al. J Mol. Neuro. 17:101-118 (2001). Thus, according to this embodiment an individual suspected of having or diagnosed with MCI is treated twice daily with a composition having from 400 mg to about 800 mg of R-flurbiprofen per dose for at least 4 weeks, at least 4 months, preferably at least 8 months, and more desirably at least 1 year. Typically, patients having MCI first complain of or have a loss of memory. Preferably an individual associated with the patient can corroborate the memory deficit. Furthermore, general cognition is not sufficiently impaired to cause concern about more widespread cognitive disorder and although daily living activities may be affected that are not significantly impaired and the patients are not demented. Individuals having or suspected of having MCI that are treated according to this embodiment can expect to slow cognitive decline and/or progression to probable AD.

### 1.3 Dosages

The invention is based on the discovery that a dosage having R-flurbiprofen in an amount of about 400 mg to about 800 mg per dose provides a PK profile believed to be effective in treating mild-to-moderate AD. Without wishing to be bound by theory, it is believed that PK profile obtained maximizes therapeutic effects while minimizing side-effects thereby providing maximum benefit to the patient. The dose can be provided twice daily, in a single or multiple dosage units (i.e., tablets or capsules) of about 350 mg R-flurbiprofen, 400 mg R-flurbiprofen, 450 mg R-flurbiprofen, 500 mg R-flurbiprofen, 550 mg R-flurbiprofen, 600 mg R-flurbiprofen, 650 mg R-flurbiprofen, 700 mg R-flurbiprofen, 750 mg R-flurbiprofen, 800 mg R-flurbiprofen, or 850 mg R-flurbiprofen. Preferably, the dose is 400 mg; thus, a preferred composition of the invention comprises 400 mg R-flurbiprofen and a carrier or vehicle suitable for oral administration, e.g., in tablets or capsules. Another preferred dose is 800 mg of R-flurbiprofen, and a preferred composition of the invention comprises 400 mg R-flurbiprofen and a carrier or vehicle suitable for oral administration, e.g., in tablets or capsules. Preferably, the compositions are substantially free of S-flurbiprofen.

In one embodiment, oral administration of a single dose to a fasting subject, provides a Cₘₐₓ of about 25-150 μg per mL per dose, and, preferably, between 30-95 μg per mL per dose. Administration of a single dose of the compositions of the invention to a fasting subject provides an AUC (area under curve of concentration versus time; total drug exposure) of from about 200 hr. g/mL to about 600 hr·μg/mL. Preferably, the tₘₐₓ (time to Cₘₐₓ) is from about 0.50 to 3.75 hours, or is from about 0.75 hour to about 3 hours, or is from about 1.00 to about 3.75 hours. Preferably, tₘₐₓ is achieved at about 2 hours after administration. Preferably, the t_{1/2} (half-life) is from about 3.75 to about 8.5 hours. Alternatively, a low dose regimen provides R-flurbiprofen to the individual in a dosage of about 200 mg. A low dose regimen can, for example, be used after the dosing regimen of 400 to 800 b.i.d.

Oral administration of a dose, twice daily for at least 4 months, preferably 8 months, and more preferably 1 year, provides an improvement or lessening of decline in cognitive function, biochemical disease marker progression, and/or plaque pathology.

Desirably, the composition of the invention are substantially free of the S-stereoisomer of flurbiprofen. In one aspect, substantially free of the S-stereoisomer means at least 90% by weight R-flurbiprofen to 10% by weight or less of S-flurbiprofen of the total flurbiprofen (S+R flurbiprofen) in said pharmaceutical composition. In another aspect, substantially free of the S-stereoisomer means at least 95% by weight R-flurbiprofen to 5% by weight or less of S-flurbiprofen of the total flurbiprofen (S+R flurbiprofen) in the pharmaceutical composition. In yet another aspect, substantially free of the S-stereoisomer means at least 99% by weight R-flurbiprofen to 1% by weight or less of S-flurbiprofen of the total flurbiprofen (S+R flurbiprofen) in the pharmaceutical composition. In yet another aspect, substantially free of the S-stereoisomer means at least 99.9% by weight R-flurbiprofen to 0.1% by weight or less of S-flurbiprofen of the total flurbiprofen (S+R flurbiprofen) in the pharmaceutical composition. In one aspect, a preferred dosage form is a tablet. In another aspect, a preferred dosage form is a capsule. In other aspects, the composition provides an improvement or lessening in decline in biochemical disease marker progression, plaque pathology, quality of life indicators or combinations of any disease parameters.

The decline in cognitive function can be characterized by cognition tests. It is preferred that the lessening in decline in cognitive function is at least 25% as compared to individuals treated with placebo, more preferably at least 40%, and even more preferably at least 60%. For example, an individual treated with placebo having probably mild-to-moderate Alzheimer's disease is expected to score approximately 5.5 points higher on the ADAS-cog test after a specified period of time (e.g., 1 year) whereas an individual treated with a composition of the invention for the same period of time will score only approximately 3.3 points higher on the ADAS-cog scale, i.e., will show 60% of the decline in cognitive function relative to untreated individuals, or 2.2 points higher i.e., will show 40% of the decline in cognitive function relative to untreated individuals, when treated for the same specified period of time.

In a specific embodiment of this aspect of the invention, the dosage is provided as a pharmaceutical composition that is composed of R-flurbiprofen, a pharmaceutically acceptable salt, a release agent, and additional optional ingredients. In another specific embodiment of this aspect of the invention, the dosage is provided as a pharmaceutical composition that is a tablet composed of R-flurbiprofen, microcrystalline cellulose, colloidal silicon dioxide, and magnesium stearate. In another specific embodiment of this aspect of the invention, the dosage is provided as a pharmaceutical composition that is a capsule is composed of R-flurbiprofen, microcrystalline cellulose, colloidal silicon dioxide, and magnesium stearate, all encapsulated in lactose monohydrate, hydroxyl propyl methyl cellulose, titanium dioxide, tracetin/glycerol triacetate, and iron oxide.

### 1.4 PK Profile

The present invention provides for the administration of R-flurbiprofen to an individual, for example, and individual having mild to moderate AD, so as to obtain a desired pharmacokinetic profile, for example, a desired concentration of R-flurbiprofen in the plasma over a period of time. Such preferred pharmacokinetic profiles and/or endpoints may be achieved through the administration of specific doses, for example, 400 mg or 800 mg once or twice a day, or may be achieved through the administration of doses individually-tailored for the specific recipient, taking into account factors such as weight, percent body fat, metabolism, ingestion of NSAIDs, etc.

Thus, in one embodiment, the invention provides for a method of administering R-flurbiprofen to an individual, wherein said R-flurbiprofen is administered in an amount sufficient to result in a plasma Cₘₐₓ of about 25 to about 150 μg per mL, and wherein said individual is known to have, or is suspected of having, AD. In a more specific embodiment, said plasma Cₘₐₓ is from about 30 to about 95 μg per mL. In another more specific embodiment, said Cₘₐₓ is from about 40 to about 80 μg per mL. In another embodiment, said Cₘₐₓ is between about 100 and about 600 μM. In a more specific embodiment, said plasma Cₘₐₓ is from about 160 to about 380 μM. In another more specific embodiment, said Cₘₐₓ is from about 170 to about 240 μM. In a specific, preferred embodiment, said individual has mild to moderate AD.

In another embodiment, the invention provides for a method of administering R-flurbiprofen to an individual, wherein said R-flurbiprofen is administered in an amount sufficient to result in a cerebrospinal fluid Cₘₐₓ of about 0.05 to about 7.5 μg per mL, and wherein said individual is known to have, or is suspected of having, AD. In another embodiment, said Cₘₐₓ is from about 0.08 to about 4.5 μg per mL. In another embodiment, the invention provides for a method of administering R-flurbiprofen to an individual, wherein said R-flurbiprofen is administered in an amount sufficient to result in a cerebrospinal fluid Cₘₐₓ of about 2 to 30 μM; from about 3.2 μM to about 20 μM; or from about 4 μM to about 12 μM.

The time to achieve plasma Cₘₐₓ will depend upon the individual to be treated, but is preferably between 0.75 to 3.75 hours. In various preferred embodiments, the tₘₐₓ (time to Cₘₐₓ) is from about 1.0 to 3.75 hours, or is from about 1.00 hour to about 3 hours, or is from about 1.00 to about 2.5 hours. Preferably, tₘₐₓ is about 2 hours after administration. Preferably, the t_{1/2} (half-life) is from about 3.75 to about 8.5 hours.

Somewhat more time is expected to achieve a cerebrospinal fluid Cₘₐₓ; however, this Cₘₐₓ is expected to be achieved between about 1 hour and about 6 hours after administration of a dose of R-flurbiprofen according to the invention.

R-flurbiprofen levels in the plasma or in the cerebrospinal fluid may be assessed by any art-accepted method. Determination of the concentration of R-flurbiprofen in cerebrospinal fluid may be accomplished as follows. Cerebrospinal fluid containing flurbiprofen and an internal standard, for example, flurbiprofen-D₃, is mixed with mobile phase and centrifuged. The supernatant is then transferred to a 96-well block and an aliquot of extract is injected onto a Micromass Ultima LC-MS-MS equipped with an enantio-selective column. Peak area of the m/z 243→199 flurbiprofen product ion is measured against the peak area of the m/z 246→202 flurbiprofen-D₃ internal standard product ion. Quantification may be performed using a weighted (1/x²) linear least squares regression analysis for each enantiomer generated from fortified plasma standards prepared in bulk and frozen.

The plasma half-life will also depend upon the individual to be treated. Preferably, the plasma half-life is from about 3.75 to about 8.5 hours. Preferably, administration of a single dose to a fasting subject provides an AUC (area under curve of concentration versus time; total drug exposure) of from about 200 hr·μg/mL to about 600 hr·μg/mL. Thus, in one embodiment, the invention provides a method of administering R-flurbiprofen to an individual having one or more indications of Alzheimer's disease, wherein said administration achieves a plasma concentration in said individual of R-flurbiprofen of between 30 and 95 μg per mL by no more than 3.75 hours after administration. In a specific embodiment, said plasma concentration is achieved within 1.75 hours after administration. In another specific embodiment, said plasma concentration is achieved between 0.75 hours and 3.75 hours after administration. In another specific embodiment, said plasma concentration is between 50 and 80 μg per mL. In another specific embodiment, said individual is an individual that has been diagnoses having mild to moderate Alzheimer's disease, or that would be diagnosed as having mild to moderate Alzheimer's disease according to a test of cognition.

In another embodiment, the invention provides a method of administering R-flurbiprofen to an individual in need of improvement in one more measures of cognition, comprising administering R-flurbiprofen orally and in a manner in which plasma levels of between 30 and 95 μg per mL are reached by 3.75 hours after administration. Further, the invention encompasses repeated dosing to achieve these levels for 1 week, two weeks, three weeks, four weeks, one month, two months, three months, four months, five months, six months, seven months, eight months, nine months, ten months, eleven months, one year, or preferably more than one year.

In another embodiment, the invention provides a method of treating an individual having, or suspected of having, Alzheimer's disease, comprising administering R-flurbiprofen in an amount sufficient to result in a Cₘₐₓ of about 30 to about 95 μg per mL. In a more specific embodiment, said Cₘₐₓ is between 40 and 80 μg per mL. The invention further provides a method of reducing a decline in a measure of cognitive function of an individual comprising administering R-flurbiprofen to said individual, wherein said administering results in the reduction of the decline in said measure of cognitive function as compared to a control. In a specific embodiment, said control is the decline in said measure of cognitive function in an individual not given R-flurbiprofen, wherein said individual has or is suspected of having Alzheimer's disease. In another specific embodiment, said control is the average decline in said measure of cognitive function in a plurality of individuals not given flurbiprofen, wherein said individuals have or are suspected of having Alzheimer's disease. In another specific embodiment, said reduction in said decline in said measure of cognitive function is at least 25% compared to said control. In another specific embodiment, said reduction in said decline in said measure of cognitive function is at least 40% compared to said control. In another specific embodiment, said reduction in said decline in said measure of cognitive function is at least 60% compared to said control. In another specific embodiment, said measure of cognitive function is an ADAS-cog test. In a more specific embodiment, said decline is 2.2 points in the ADAS-cog test over one year. In another more specific embodiment, said decline is 3.3 points in the ADAS-cog test over one year. In another specific embodiment, said R-flurbiprofen is administered in a dose of about 400 mg twice daily. In another specific embodiment, said R-flurbiprofen is administered in a dose of about 800 mg twice daily.

In another embodiment, the invention provides for a method of improving, or lessening a decline in, the progression of one or more disease markers of Alzheimer's disease in an individual having, or suspected of having, Alzheimer's disease, comprising administering R-flurbiprofen to said individual. In a specific embodiment, said R-flurbiprofen is administered in an amount that achieves a Cₘₐₓ of about 30 to about 95 μg per mL. In a more specific embodiment, said Cₘₐₓ is between 40 and 80 μg per mL. In a specific embodiment, said administration is continued at least once a day for at least four months. In another specific embodiment, said administration is continued at least once a day for at least eight months. In another specific embodiment, said administration is continued at least once a day for at least twelve months. In a specific embodiment, said disease marker is amyloid beta peptide (Aβ), Aβ₄₂, or tau. In another specific embodiment, said R-flurbiprofen is administered in a dose of about 400 mg twice daily. In another specific embodiment, said R-flurbiprofen is administered in a dose of about 800 mg twice daily.

In another embodiment, the invention provides for a method of improving, or lessening a decline in, plaque pathology associated with Alzheimer's disease in an individual having, or suspected of having, Alzheimer's disease, comprising administering R-flurbiprofen to said individual. In a specific embodiment, said administration of R-flurbiprofen achieves a Cₘₐₓ of about 30 to about 95 μg per mL. In a more specific embodiment, said Cₘₐₓ is between 40 and 80 μg per mL. In a specific embodiment, said administration is continued at least once a day for at least four months. In another specific embodiment, said administration is continued at least once a day for at least eight months. In another specific embodiment, said R-flurbiprofen is administered in a dose of about 400 mg per day. In another specific embodiment, said R-flurbiprofen is administered in a dose of about 800 mg per day.

In one embodiment, the invention provides for a method of administering R-flurbiprofen to an individual, wherein said R-flurbiprofen is administered in an amount sufficient to result in a plasma Cₘₐₓ of about 35 to about 50 μg per mL, and wherein said individual is known to have, or is suspected of having, AD. In a more specific embodiment, said plasma Cₘₐₓ is from about 38 to about 48 μg per mL. In another more specific embodiment, said Cₘₐₓ is from about 39 to about 46 μg per mL. In another embodiment, the invention provides for a method of administering R-flurbiprofen to an individual, wherein said R-flurbiprofen is administered in an amount sufficient to result in a plasma Cₘₐₓ of about 45 to about 58 μg per mL, and wherein said individual is known to have, or is suspected of having, AD. In a more specific embodiment, said plasma Cₘₐₓ is from about 47 to about 56 μg per mL. In a more specific embodiment, said plasma Cₘₐₓ is from about 48 to about 55 μg per mL. In a specific, preferred embodiment, said individual has mild to moderate AD. In another specific, preferred embodiment, said individual has MCI.

In another embodiment, the time to achieve plasma Cₘₐₓ will depend upon the individual to be treated, but is preferably between 0.75 to 2.25 hours. In various preferred embodiments, the tₘₐₓ (time to Cₘₐₓ) is from about 1.0 to 2.1 hours, or is from about 1.25 hour to about 2 hours, or is from about 1.00 to about 2.5 hours. Preferably, the t_{1/2} (half-life) is from about 6.00 to about 10.0 hours; more preferably from about 6.5 to about 9.5 hours; and more preferably from about 7 to about 9 hours. Preferably the AUC (area under the curve; total drug exposure) is from about 350 (hr*ug/mL) to 750 (hr*ug/mL); is from about 400 (hr*ug/mL) to 650 (hr*ug/mL); or is from about 450 (hr*ug/mL) to 700 (hr*ug/mL). In a specific, preferred embodiment, said individual has mild to moderate AD. In another specific, preferred embodiment, said individual has MCI.

In yet another embodiment, the time to achieve plasma Cₘₐₓ will depend upon the individual to be treated, but is preferably between 0.25 to 2.00 hours. In various preferred embodiments, the tₘₐₓ (time to Cₘₐₓ) is from about 0.25 to 1.75 hours, or is from about 0.50 hour to about 1.75 hours, or is from about 0.5 to about 1.25 hours. Preferably, the t_{1/2} (half-life) is from about 3.5 to about 8.5 hours; more preferably from about 4.0 to about 8.0 hours; and more preferably from about 4.8 to about 7.5 hours. Preferably the AUC (area under the curve; total drug exposure) is from about 250 (hr*ug/mL) to 700 (hr*ug/mL); is from about 300 (hr*ug/mL) to 650 (hr*ug/mL); or is from about 350 (hr*ug/mL) to 600 (hr*ug/mL). In a specific, preferred embodiment, said individual has mild to moderate AD. In another specific, preferred embodiment, said individual has MCI.

### 1.5 Methods of Prevention and Treatment

In an embodiment, the invention provides a method of treating Alzheimer's disease comprising administering to a patient in need of such treatment, a dose of a pharmaceutical composition comprising an effective amount of R-flurbiprofen and one or more pharmaceutically acceptable excipients, wherein a dose of an effective amount upon oral administration to a fasting subject provides a Cₘₐₓ of about 30-95 μg per mL per dose. Oral administration of a dose, twice daily for at least 4 months, more preferably 8 months, and more preferably 1 year, provides an improvement or lessening in decline in cognitive function, biochemical disease marker progression, and/or plaque pathology. The dose can be provided twice daily, in a single or multiple dosage units (i.e., tablets or capsules) where the dose is about 350 mg R-flurbiprofen, 400 mg R-flurbiprofen, 450 mg R-flurbiprofen, 500 mg R-flurbiprofen, 550 mg R-flurbiprofen, 600 mg R-flurbiprofen, 650 mg R-flurbiprofen, 700 mg R-flurbiprofen, 750 mg R-flurbiprofen, 800 mg R-flurbiprofen, or 850 mg R-flurbiprofen. Preferably, the dose is 400 mg; thus, a preferred method uses a composition of the invention comprising 400 mg R-flurbiprofen and a carrier or vehicle suitable for oral administration, e.g., in tablets or capsules. Another preferred dose is 800 mg of R-flurbiprofen, and a preferred method uses a composition of the invention comprising 400 mg R-flurbiprofen and a carrier or vehicle suitable for oral administration, e.g., in tablets or capsules. Alternatively, the invention provides a low dose based treatment regimen wherein the dose has about 200 mg R-flurbiprofen. Desirably, the composition is substantially free of the (S)-stereoisomer of flurbiprofen.

In another embodiment, the invention provides a method of preventing the onset of Alzheimer's disease comprising administering to a patient, in need of such treatment, a pharmaceutical composition comprising an effective amount of R-flurbiprofen and one or more pharmaceutically acceptable excipients, wherein a single dose of an effective amount upon oral administration to a fasting subject provides a Cₘₐₓ of about 30-95 μg per mL per dosage unit and wherein upon oral administration of a dosage unit, twice daily for at least 4 months, more preferably 8 months, and more preferably 1 year, provides an improvement or lessening of in decline in cognitive function, biochemical disease marker progression, and/or plaque pathology. Preferably, administration of a dose to a fasting subject provides an AUC (area under curve of concentration versus time; total drug exposure) of from about 200 hr·μg/mL to about 600 hr·μg/mL. Preferably, the tₘₐₓ (time to Cₘₐₓ) is from about 0.75 to 3.75 hours. Preferably, the t_{1/2} (half-life) is from about 3.75 to about 8.5 hours. The dose can be provided twice daily, in a single or multiple dosage units (i.e., tablets or capsules) where the dose is about 350 mg R-flurbiprofen, 400 mg R-flurbiprofen, 450 mg R-flurbiprofen, 500 mg R-flurbiprofen, 550 mg R-flurbiprofen, 600 mg R-flurbiprofen, 650 mg R-flurbiprofen, 700 mg R-flurbiprofen, 750 mg R-flurbiprofen, 800 mg R-flurbiprofen, or 850 mg R-flurbiprofen. Preferably, the dose is 400 mg; thus, a preferred method uses a composition of the invention comprising 400 mg R-flurbiprofen and a carrier or vehicle suitable for oral administration, e.g., in tablets or capsules. Another preferred dose is 800 mg of R-flurbiprofen, and a preferred method uses a composition of the invention comprising 400 mg R-flurbiprofen and a carrier or vehicle suitable for oral administration, e.g., in tablets or capsules. Alternatively, the invention provides a low dose based prevention regimen wherein the dose has about 200 mg R-flurbiprofen. Desirably, the composition is substantially free of the (S)-stereoisomer of flurbiprofen.

In yet another embodiment, the invention provides a method of decelerating the onset of Alzheimer's disease comprising administering to a patient in need of such treatment a pharmaceutical dosage having an effective amount of R-flurbiprofen and one or more pharmaceutically acceptable excipients, wherein a single dose of an effective amount upon oral administration to a fasting subject provides a Cₘₐₓ of about 40-95 μg per mL per dose and wherein upon oral administration of a dose, twice daily for at least 4 months, preferably 8 months, and more desirably 1 year, provides an improvement or lessening of decline in cognitive function, biochemical disease marker progression, and/or plaque pathology. Preferably, administration of a single dose to a fasting subject provides an AUC (area under curve of concentration versus time; total drug exposure) of from about 200 hr·μg/mL to about 600 hr·μg/mL. Preferably, the tₘₐₓ (time to Cₘₐₓ) is from about 0.75 to 3.75 hours. Preferably, the t_{1/2} (half-life) is from about 3.75 to about 8.5 hours. The dose can be provided twice daily, in a single or multiple dosage units (i.e., tablets or capsules) where the dose has about 350 mg R-flurbiprofen, 400 mg R-flurbiprofen, 450 mg R-flurbiprofen, 500 mg R-flurbiprofen, 550 mg R-flurbiprofen, 600 mg R-flurbiprofen, 650 mg R-flurbiprofen, 700 mg R-flurbiprofen, 750 mg R-flurbiprofen, 800 mg R-flurbiprofen, or 850 mg R-flurbiprofen. Preferably, the dose is 400 mg; thus, a preferred method uses a composition of the invention comprising 400 mg R-flurbiprofen and a carrier or vehicle suitable for oral administration, e.g., in tablets or capsules. Another preferred dose is 800 mg of R-flurbiprofen, and a preferred method uses a composition of the invention comprising 400 mg R-flurbiprofen and a carrier or vehicle suitable for oral administration, e.g., in tablets or capsules. Alternatively, the invention provides a low dose based prevention regimen wherein the dose has about 200 mg R-flurbiprofen. Desirably, the composition is substantially free of the (S)-stereoisomer of flurbiprofen.

The compounds of the invention, and dosage forms of the invention, described herein, may be administered once, twice, three times, four times or more per day.

It was discovered that tablets give an unexpectedly improved pharmacokinetic profile over capsules having the same amount of R-flurbiprofen. It was discovered that the Cₘₐₓ was lower for tablets and the peak was broader giving an improved delivery of drug for treating Alzheimer's disease as compared to capsule dosage forms.

Another discovery of the present invention that has led to the unexpected finding that doses of about 400 mg to about 800 mg R-flurbiprofen for treating (and preventing) mild-to-moderate AD is that maximal improvements in reducing or lessening decline in plaque pathology as assessed in model organisms and cell systems are seen over this range of active ingredient, above and below this unexpected range there is less of a reduction in the rate of decline in indicators of plaque pathology. Furthermore, above this range toxicity problems become a concern.

The pharmacokinetic parameters referred to herein are based on the averages for a group of about 12 individuals for each dosing regimen (12 individuals treated with 200 mg BID, 12 individuals treated with 400 mg BID, 12 individuals treated with 800 mg BID, and 12 individuals treated with placebo). The skilled artisan understands that individuals will vary and can have pharmacokinetic parameters outside the given ranges. Similarly, the efficacy or therapeutic endpoint parameters are based on averages for a group of individuals and individuals experience efficacies that fall outside the given ranges.

In another embodiment of the invention, the invention relates to a method for improving cognitive function. More particularly, this embodiment of the invention provides a method for improving cognitive function in individuals experiencing cognitive decline such as that experienced by Alzheimer's disease patients or those with mild cognitive impairment (MCI). The invention is based on the discovery that Alzheimer's disease patients that have experienced cognitive decline as a result of the disease can experience an improvement in cognition when administered a cognition improving effective amount of a pharmaceutical composition having R-flurbiprofen as the active ingredient. In one embodiment, the invention provides a method for improving cognitive function in individuals experiencing cognitive decline. According to this method, an individual in need of or desiring treatment (e.g., a patient having Alzheimer's disease or mild cognitive impairment), is administered a composition having R-flurbiprofen in an amount of about 100 mg to about 1800 mg per day for at least 4 weeks, preferably at least 4 months, and more preferably at least 6 months. Preferably, the amount of R-flurbiprofen administered to the individual is from about 200 to 1800 mg per day, more preferably the amount is from about 350 to 1650 mg per day. The composition used in the invention is formulated with one or more pharmaceutically acceptable excipients, salts, or carriers. The pharmaceutical composition can be delivered orally, preferably in a tablet or capsule dosage form. Oral administration of a single dose of the cognition improving effective amount of R-flurbiprofen to a fasting subject provides a Cₘₐₓ of about 30-95 μg per mL. Oral administration of the R-flurbiprofen composition twice daily (b.i.d) for at least 4 weeks, preferably at least 4 months, even more preferably at least 6 months, and more desirably at least 1 year, provides an improvement in cognitive function as characterized by cognition tests. It is preferred that the improvement in cognitive function is statistically significant as compared to individuals treated with placebo. For example, an individual treated with placebo having probable mild-to-moderate Alzheimer's disease is expected to score approximately 5.5 points lower on the ADAS-cog test after a specified period of time of treatment (e.g., 1 year) whereas an individual (having mild to moderate Alzheimer's disease) treated with the R-flurbiprofen composition for the same period of time will score no higher on the ADAS-cog scale or will have a better, i.e. lower, score (e.g., 0.25, 0.5, 0.75, 1.0, 1.5, 2.0, 2.5, 3.0, or 4.0 or more points better). Desirably, the oral dose is provided in capsule or tablet form. In a specific embodiment of this aspect of the invention, the dosage is provided as a pharmaceutical composition composed of R-flurbiprofen, a pharmaceutically acceptable salt, a release agent, and optionally additional ingredients.

In another embodiment of the invention, the invention relates to a method for improving performance on cognitive tests. More particularly, this embodiment of the invention provides a method for improving performance on the ADAS-cog test in individuals who have experienced cognitive decline such as that experienced by Alzheimer's disease patients or those with mild cognitive impairment. The invention is based on the discovery that individuals that have experienced cognitive decline as a result of a disease or condition such as Alzheimer's disease or mild cognitive impairment can improvement their performance on the ADAS-cog test when administered a cognition improving effective amount of a pharmaceutical composition having R-flurbiprofen as the active ingredient. According to this method, an individual in need of or desiring treatment is identified and given the ADAS-cog test. The individual is then treated with a composition having R-flurbiprofen in an amount of about 100 mg to about 1800 mg per day for at least 4 weeks, preferably at least 4 months, and more preferably at least 6 months. Preferably, the amount of R-flurbiprofen administered to the individual is from about 200 to 1800 mg per day, more preferably the amount is from about 350 to 1650 mg per day. An individual who is treated according to this method is then given the ADAS-cog test and the individual is expected to improve performance on the test. By improving performance, it is meant that a group of individuals that underwent the treatment will score the same or better (i.e., lower), in a statistically significant manner, on the ADAS-cog test. Preferably, the improvement is 0.25, 0.5, 0.75, 1.0, 1.5, 2.0, 3.0, 4.0 or more points better (i.e., lower) on the ADAS-cog test. The composition used in the invention is formulated with one or more pharmaceutically acceptable excipients, salts, or carriers. The pharmaceutical composition can be delivered orally, preferably in a tablet or capsule dosage form. Oral administration of a single dose of the cognition improving effective amount of R-flurbiprofen to a fasting subject provides a Cₘₐₓ of about 30-95 μg per mL. Oral administration of the R-flurbiprofen composition twice daily (b.i.d) for at least 4 weeks, preferably at least 4 months, even more preferably at least 6 months, and more desirably at least 1 year, provides an improvement in performance on the ADAS-cog test. It is preferred that the improvement in performance on the ADAS-cog test is statistically significant as compared to individuals treated with placebo. For example, an individual treated with placebo having probable mild-to-moderate Alzheimer's disease is expected to score approximately 5.5 points higher on the ADAS-cog test after a specified period of time of treatment (e.g., 1 year) whereas an individual (having mild to moderate Alzheimer's disease) treated with the R-flurbiprofen composition for the same period of time will score no higher on the ADAS-cog scale or will have a better, i.e. lower, score (e.g., 0.25, 0.5, 0.75, 1.0, 1.5, 2.0, 2.5, 3.0, or 4.0 or more points better). Desirably, the oral dose is provided in capsule or tablet form. In a specific embodiment of this aspect of the invention, the dosage is provided as a pharmaceutical composition composed of R-flurbiprofen, a pharmaceutically acceptable salt, a release agent, and optionally additional ingredients.

### 1.6 Pharmaceutical Formulations

The pharmaceutical compositions and dosages of the present invention may be administered in any pharmaceutically-acceptable manner; however, tablet and capsule forms are preferred. However, for the dosages of R-flurbiprofen described herein, tablets give an unexpectedly improved pharmacokinetic profile over capsules having the same amount of R-flurbiprofen. This is because the Cₘₐₓ is lower for tablets and the peak is broader giving an improved delivery of drug for treating Alzheimer's disease as compared to capsule dosage forms.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

Soft gelatin capsules can be prepared in which capsules contain a mixture of the active ingredient and vegetable oil or non-aqueous, water miscible materials such as, for example, polyethylene glycol and the like. Hard gelatin capsules may contain granules of the active ingredient in combination with a solid, pulverulent carrier, such as, for example, lactose, saccharose, sorbitol, mannitol, potato starch, corn starch, amylopectin, cellulose derivatives, or gelatin.

Tablets are the preferred dosage form because of the improved pharmacokinetic profile as compared with other dosage forms (see above), and because of advantages afforded both to the patient (e.g., accuracy of dosage, compactness, portability, blandness of taste as well as ease of administration) and to the manufacturer (e.g., simplicity and economy of preparation, stability as well as convenience in packaging, shipping and dispensing). Tablets are solid pharmaceutical dosage forms containing therapeutic drug substances with or without suitable additives.

Tablets are typically made by molding, by compression or by generally accepted tablet forming methods. Accordingly, compressed tablets are usually prepared by large-scale production methods while molded tablets often involve small-scale operations.

Tablets for oral use are typically prepared in the following manner, although other techniques may be employed. The solid substances are ground or sieved to a desired particle size, and the binding agent is homogenized and suspended in a suitable solvent. The active ingredient and auxiliary agents are mixed with the binding agent solution. The resulting mixture is moistened to form a uniform suspension. The moistening typically causes the particles to aggregate slightly, and the resulting mass is gently pressed through a stainless steel sieve having a desired size. The layers of the mixture are then dried in controlled drying units for determined length of time to achieve a desired particle size and consistency. The granules of the dried mixture are gently sieved to remove any powder. To this mixture, disintegrating, anti-friction, and anti-adhesive agents are added. Finally, the mixture is pressed into tablets using a machine with the appropriate punches and dies to obtain the desired tablet size. The operating parameters of the machine may be selected by the skilled artisan.

In general, there are three general methods of tablet preparation: (1) the wet-granulation method; (2) the dry-granulation method; and (3) direct compression. These methods are well known to those skilled in the art. See, Remington's Pharmaceutical Sciences, 16th and 18th Eds., Mack Publishing Co., Easton, Pa. (1980 and 1990). See, also, U.S. Pharmacopeia XXI, U.S. Pharmacopeial Convention, Inc., Rockville, Md. (1985).

Various tablet formulations may be made in accordance with the present invention. These include tablet dosage forms such as sugar-coated tablets, film-coated tablets, enteric-coated tablets, multiple-compressed tablets, prolonged action tablets and the like. Sugar-coated tablets (SCT) are compressed tablets containing a sugar coating. Such coatings may be colored and are beneficial in covering up drug substances possessing objectionable tastes or odors and in protecting materials sensitive to oxidation. Film-coated tablets (FCT) are compressed tablets that are covered with a thin layer or film of a water-soluble material. A number of polymeric substances with film-forming properties may be used. The film coating imparts the same general characteristics as sugar coating with the added advantage of a greatly reduced time period required for the coating operation. Enteric-coated tablets are also suitable for use in the present invention. Enteric-coated tablets (ECT) are compressed tablets coated with substances that resist dissolution in gastric fluid but disintegrate in the intestine. Enteric coating can be used for tablets containing drug substances that are inactivated or destroyed in the stomach, for those which irritate the mucosa or as a means of delayed release of the medication.

Multiple compressed tablets (MCT) are compressed tablets made by more than one compression cycle, such as layered tablets or press-coated tablets. Layered tablets are prepared by compressing additional tablet granulation on a previously compressed granulation. The operation may be repeated to produce multilayered tablets of two, three or more layers. Typically, special tablet presses are required to make layered tablets. See, for example, U.S. Pat. No. 5,213,738, incorporated herein in its entirety by reference thereto.

Press coated tablets are another form of multiple compressed tablets. Such tablets, also referred to as dry-coated tablets, are prepared by feeding previously compressed tablets into a tableting machine and compressing another granulation layer around the preformed tablets. These tablets have all the advantages of compressed tablets, i.e., slotting, monogramming, speed of disintegration, etc., while retaining the attributes of sugar coated tablets in masking the taste of the drug substance in the core tablet. Press-coated tablets can also be used to separate incompatible drug substances. Further, they can be used to provide an enteric coating to the core tablets. Both types of tablets (i.e., layered tablets and press-coated tablets) may be used, for example, in the design of prolonged-action dosage forms of the present invention.

Pharmaceutical compositions or unit dosage forms of the present invention in the form of prolonged-action tablets may comprise compressed tablets formulated to release the drug substance in a manner to provide medication over a period of time. There are a number of tablet types that include delayed-action tablets in which the release of the drug substance is prevented for an interval of time after administration or until certain physiological conditions exist. Repeat action tablets may be formed that periodically release a complete dose of the drug substance to the gastrointestinal fluids. Also, extended release tablets that continuously release increments of the contained drug substance to the gastrointestinal fluids may be formed.

In practical use, optically pure R(−)-flurbiprofen can be combined as the active ingredient in intimate admixture with a pharmaceutically acceptable carrier according to conventional pharmaceutical compounding techniques. The pharmaceutically acceptable carrier may take a wide variety of forms depending on the form of preparation desired for administration, e.g., oral, parenteral (including intravenous, subcutaneous, intrathecal, and intramuscular), transdermal, and topical. In preparing the compositions for oral dosage form, any of the usual pharmaceutical media or excipients may be employed. These include, for example, water, glycols, oils, alcohols, flavoring agents, preservatives, coloring agents and the like in the case of oral liquid preparations such as suspensions, elixirs and solutions; or aerosols; or excipients such as starches, sugars, microcrystalline cellulose, diluents, granulating agents, lubricants, binders, disintegrating agents and the like in the case of oral solid preparations such as powders, capsules, caplets, and tablets. Solid oral preparations are generally preferred over liquid ones. Because of their ease of administration, tablets and capsules represent the most advantageous oral dosage unit forms, in which case solid pharmaceutical pharmaceutically acceptable excipients are obviously employed. If desired, tablets may be coated by standard aqueous or nonaqueous techniques. Preferred solid oral preparations are tablets and capsules.

Pharmaceutical stabilizers may be used to stabilize compositions comprising optically pure R(−)-flurbiprofen, or pharmaceutically acceptable salts, solvates, or clathrates thereof. Acceptable stabilizers include, but are not limited to, L-cysteine hydrochloride, glycine hydrochloride, malic acid, sodium metabisulfite, citric acid, tartaric acid, and L-cystine dihydrochloride. See, e.g. U.S. Pat. Nos.: 5,731,000; 5,763,493; 5,541,231; and 5,358,970, all of which are incorporated herein by reference.

In addition to the common dosage forms set out above, the active ingredient (i.e., optically pure R-flurbiprofen) can be administered by controlled release means and/or delivery devices capable of releasing the active ingredient at a rate required to maintain constant pharmacological activity for a desirable period of time. Such dosage forms provide a supply of a drug to the body during a predetermined period of time and thus maintain drug levels in the therapeutic range for longer periods of time than conventional non-controlled formulations. Examples of controlled release pharmaceutical compositions and delivery devices which may be adapted for the administration of the active ingredient of the invention are described in U.S. Pat. Nos. 3,847,770; 3,916,899; 3,536,809; 3,598,123; 3,630,200; 4,008,719; 4,687,610; 4,769,027; 5,674,533; 5,059,595; 5,591,767; 5,120,548; 5,073,543; 5,639,476; 5,354,566; and 5,733,566, the disclosures of which are incorporated herein by reference.

Pharmaceutical compositions of the invention suitable for oral administration may be presented as discrete units such as capsules, cachets, caplets, or tablets or aerosol sprays, each containing a predetermined amount of the active ingredient as a powder, as granules, or as a solution or a suspension in an aqueous or non-aqueous liquid, an oil-in-water emulsion, or a water-in-oil liquid emulsion. Such compositions may be prepared by any of the methods of pharmacy which include the step of bringing into association the active ingredient with a pharmaceutically acceptable carrier which constitutes one or more necessary ingredients. In general, the compositions are prepared by uniformly and intimately admixing the active ingredient with a liquid pharmaceutically acceptable carrier or a finely divided solid pharmaceutically acceptable carrier, or both, and then, if necessary, shaping the product into the desired presentation. For example, a tablet may be prepared by compression or molding, optionally with one or more accessory ingredients. Compressed tablets may be prepared by compressing in a suitable machine the active ingredient in a free-flowing form such as powder or granules, optionally mixed with a binder, lubricant, inert diluent, disintegrating agent, and/or surface active or dispersing agent. Molded tablets may be made by molding in a suitable machine a mixture of the powdered compound moistened with an inert liquid diluent.

The dosage form of R-flurbiprofen, for example, 400 mg or 800 mg, can be compounded with any other compound determined to be suitable for the treatment of Alzheimer's disease. For example, the dose of R-flurbiprofen may be compounded with an acetylcholinesterase (AChE) inhibitor. Examples of ACHE inhibitors useful for the treatment of Alzheimer's disease include, without limitation, Galanthamine (galantamine, Reminyl); E2020 (Donepezil, Aricept); Physostigmine; Tacrine (tetrahydroaminoacridine, THA); Rivastigmine; Phenserine; Metrifonate (Promem); or Huperazine. The dose of R-flurbiprofen may also be compounded with one or more pharmaceutically-acceptable antioxidants, for example, vitamin C (for example, 500-1000 mg per dose of R-flurbiprofen) and/or vitamin E (for example, 400-800 IU per dose of R-flurbiprofen).

### 1.7 Preparation of R-Flurbiprofen

Flurbiprofen useful in making the pharmaceutical compositions and formulations of the present invention, and in useful in performing the methods of the present invention, may be made by any known method that produces optically-pure R-flurbiprofen.

R-flurbiprofen is commercially available from, e.g., Sepracor Inc., (Marlborough, Mass.).

In addition to commercial sources of R-flurbiprofen, racemic mixtures of flurbiprofen are available from a number of commercial sources including, e.g., Sigma (St. Louis, Mo.). The optically pure R-isomer flurbiprofen (or a desired enantiomeric excess of R-flurbiprofen) can then be obtained by resolving the racemic mixtures according to well-known methods.

R-flurbiprofen compositions are disclosed in, e.g., U.S. Pat. No. 5,200,198 to Geisslinger et al.

Methods of resolving R-flurbiprofen from the racemate are disclosed in U.S. Pat. No. 5,599,969 to Hardy et al. which discloses contacting the racemates with α-methylbenzylamine salt in a solvent mixture of toluene and methanol, followed by recrystallization of the diastereomer salt. The diastereomer salts are then separated to give the resolved flurbiprofen enantiomers. U.S. Pat. No. 4,209,638 to Boots Co. discloses a process for resolving 2-arylproprionic acids which include flurbiprofen by mixing the racemate with a chiral organic nitrogenous base under certain conditions followed by recovery and separation of the diastereomeric salts. Other patents disclosing processes for resolving racemic arylproprionic acids include U.S. Pat. Nos. 4,983,765 to PAZ; 5,015,764 to Ethyl Corp.; 5,235,100 to Ethyl Corp.; 5,574,183 to Albemarle Corp.; 5,510,519 to Sumitomo Chemical Company.

Methods of tableting R-flurbiprofen and arylproprionic acids are disclosed in, e.g., U.S. Pat. No. 5,667,807 to Humer et al.; U.S. Pat. No. 5,565,613 to Geisslinger at al.; U.S. Pat. No. 6,471,991 to Robinson et al.; and U.S. Pat. No. 6,379,707 to Vladyka et al.

### 2. EXAMPLES

### 2.1 Example 1

### R-Flurbiprofen Containing Tablets

| Ingredient | Amount | Preferred Ranges |
|---|---|---|
| R-Flurbiprofen | 400 mg | +20% to −20% |
| Microcrystalline Cellulose | 392 mg | +20% to −20% |
| Colloidal Silicon Dioxide | 4 mg | +50% to −50% |
| Magnesium Stearate | 4 mg | +50% to −50% |

The tablets are prepared using art known procedures.

### 2.2 Example 2

### R-Flurbiprofen Containing Coated Tablets

| Ingredient | Amount | Preferred Ranges |
|---|---|---|
| R-Flurbiprofen | 400 mg | +20% to −20% |
| Microcrystalline Cellulose | 392 mg | +20% to −20% |
| Colloidal Silicon Dioxide | 4 mg | +50% to −50% |
| Magnesium Stearate | 4 mg | +50% to −50% |
| Coated with | | |
| Lactose monohydrate | | |
| Hydroxyl propyl methyl | | |
| cellulose | | |
| Titanium dioxide | | |
| Tracetin/glycerol triacetate | | |
| Iron oxide | | |

The coated tablets are produced using art known procedures.

### 2.3 Example 3

### R-Flurbiprofen Capsules

| Ingredient | Amount | Preferred Ranges |
|---|---|---|
| R-Flurbiprofen | 400 mg | +20% to −20% |
| Microcrystalline Cellulose | 392 mg | +20% to −20% |
| Colloidal Silicon Dioxide | 4 mg | +50% to −50% |
| Magnesium Stearate | 4 mg | +50% to −50% |
| Encapsulated in gelatin | | |

The capsules are produced using art known procedures.

### 2.4 Example 4

### R-Flurbiprofen Tablets

| Ingredient | Amount | Preferred Ranges |
|---|---|---|
| R-Flurbiprofen | 200 mg | +20% to −20% |
| Microcrystalline Cellulose | 196 mg | +20% to −20% |
| Colloidal Silicon Dioxide | 2 mg | +50% to −50% |
| Magnesium Stearate | 2 mg | +50% to −50% |

### 2.5 Example 5

### Clinical Investigation of R-Flurbiprofen

A clinical investigation to demonstrate its efficacy in lowering the observed levels of the exploratory biomarker Aβ42 in plasma and cerebrospinal fluid (CSF), i.e. a Phase I study of the safety of R-flurbiprofen, is conducted as follows.

Objective: To assess the safety and tolerability of ascending oral doses of R-flurbiprofen in healthy subjects 55 to 80 years of age; to derive pharmacokinetic parameters of R-flurbiprofen in plasma and the concentration of R-flurbiprofen in CSF, after administration of R-flurbiprofen; and to measure the plasma and CSF concentrations of Aβ42, Aβ40 and Aβ38 in subjects before and after 21 days administration of R-flurbiprofen or placebo. Normal older subjects will be able to recall and report adverse events (AEs) more reliably than patients with AD.

Clinical Hypothesis: Administration of R-flurbiprofen at one of several dose levels for 21 days to healthy subjects aged 55 to 80 years will be well tolerated and severity of AEs will not preclude subsequent trials in patients with mild-to-moderate dementia.

Experimental Plan

Study Design

The study is conducted as a double-blind, placebo-controlled, sequential, ascending dose level, multiple dose study, including men and women. Sixteen subjects are assigned to each of three sequential cohorts: 200 mg BID, 400 mg BID, and 800 mg BID of R-flurbiprofen, each with matched placebo BID. Each cohort contains a 3:1 ratio of assignment to active drug (n=12) versus placebo (n=4), resulting in 4 treatment groups of 12 subjects each after combining the 3 placebo groups. The study is balanced by site and stratified by gender. Subjects receive either R-flurbiprofen at the specified dose or a matched placebo orally for 21 days. Blood and urine samples are collected for laboratory safety data at Study Day 1, Study Day 21, and End of Study (30-Day Follow Up) visit. Blood and CSF are collected for pharmacokinetic and biomarker measures at Study Day 1 and after 21 days of treatment with the study drug.

48 subjects are enrolled in this study, including sixteen in each sequential cohort, consisting of 12 treated with R-flurbiprofen and 4 treated with placebo.

Inclusion Criteria

Subjects must meet all of the following inclusion criteria in order to participate in the study:
Men or women 55 to 80 years;
Ability to read and understand English, to ensure compliance with cognitive testing and study visit procedures.
No significant cognitive or functional impairment (Mini-Mental State Examination [MMSE] score>27/30).
Female subjects must be surgically sterile or postmenopausal for >1 year.
Be willing to limit aspirin use to cardioprotective dose levels (e.g., <100 mg aspirin per day) for the duration of the study.
Have adequate hepatic, renal, and hematologic function.

Exclusion Criteria
Significant neurologic disease such as Parkinson's disease, stroke, brain tumor, multiple sclerosis or seizure disorder.
Major depression in past 12 months, major mental illness such as schizophrenia, or recent (in past 12 months) alcohol or substance abuse.
History of hypersensitivity to NSAIDs including cyclooxygenase 2 (COX-2) specific inhibitors such as Celecoxib and rofecoxib.
History of upper gastrointestinal (GI) bleeds requiring a transfusion in the past three years.
Active ulcer disease diagnosed in past 12 months; this includes patients who are taking medications for upper gastrointestinal protection on a regular (daily) basis, including proton pump inhibitors, H-2 receptor antagonists, and cytoprotective agents. The following drugs are examples: rabeprazole/Aciphex®, omeprazole/Prilosec®/Nexium®, pantoprozole/Protonix®, lansoprozole/Prevacid® (proton pump inhibitors); cimetidine/Tagamet®, ranitidine/Zantac®, famotidine/Pepcid® (H-2 receptor antagonists), misoprostol/Cytotec®, sucralfate/Carafate® (cytoprotective agents).
History of NSAID-related ulcer in the past 5 years.
Use of NSAIDs or immunosuppressive drugs at any dose at a frequency greater than 7 days/month during the 2 months prior to Study Day 1.
History of, or evidence of, active malignancy, except for basal cell carcinoma and squamous cell carcinoma of the skin, within the 24 months prior to entry.
Chronic or acute renal or hepatic disorder or a significant bleeding disorder or any other condition, which in the Investigator's opinion might preclude study participation.
Contraindications to lumbar puncture (anticoagulant treatment, major structural abnormality or infection in the area of the lumbosacral spine, hypersensitivity to lidocaine).
Use of any investigational drugs or devices within 30 days, or 5 half-lives, whichever is longer, prior to screening.
Major surgery within 12 weeks prior to Study Day 1.
New York Heart Association Class III or IV.
Active systemic infections or any serious uncontrolled medical condition within 30 days.
Dementia or altered mental status.
Antiretroviral therapy for human immunodeficiency virus (HIV). Subjects who are HIV positive and are not receiving antiretroviral therapy may participate.
Anticoagulant therapy such as warfarin within 12 weeks prior to randomization.
Previous participation in this study.
Treatment with any CYP2C9 inhibitor within a 2 week period prior to randomization. The following drugs and herbal preparations are examples of CYP2C9 inhibitors: amiodarone/Cordarone®, fluconazole/Diflucan®, fluvastatin/Lescol®, fluvoxamine/Luvox®, isoniazid/INH®, lovastatin/Mevacor®, miconazole, paroxetine/Paxil®, phenylbutazone, probenicid/Benemid®, sertraline/Zoloft®, sulfamethoxazole/Gantanol®, sulfaphenazole, teniposide/Vumon®, trimethoprim/Bactrim®, zafirlukast/Accolate®; danshen (Salvia miltiorrhiza); Lycium barbarum.
Use of herbal preparations prohibited by the investigator. Controlled clinical trials have not been conducted with herbal preparations. Therefore, the possibility of interaction with the study drug can not be ruled out. Use of herbal preparations will be closely monitored and recorded by the investigator, and prohibited at his or her discretion.

Treatment Procedures

Drug Dosage, Administration, and Schedule

The drug dosage and administration schedule is summarized in the table below. Subjects are instructed to take 1 capsule from Bottle A and 1 capsule from Bottle B 2 times per day (BID) for 20 days; Bottles A and B may contain R-flurbiprofen or placebo. The intraday dosing interval is approximately 12 hours, and the study drug is taken at approximately the same time each day. After 20 days (i.e., on Study Day 21), study participants undergo a pharmacokinetic study, as described below. Daily doses for the 4 groups of the study are:
400 mg R-flurbiprofen (given as one 200 mg capsule and 1 placebo capsule BID)
800 mg R-flurbiprofen (given as one 400 mg capsule and 1 placebo capsule BID)
1600 mg R-flurbiprofen (given as two 400 mg capsules BID)

Placebo (given as 2 placebo capsules BID)

TABLE
Treatment groups and dosing schedule

| | Treatment Groups | | | |
|---|---|---|---|---|
| | Placebo | 200 mg | 400 mg | 800 mg |
| Daily dose from: | BID | BID | BID | BID |
| Bottle A | One placebo | One | One | One |
| | capsule | 200 mg | 400 mg | 400 mg |
| | | capsule | capsule | capsule |
| Bottle B | One placebo | One placebo | One placebo | One |
| | capsule | capsule | capsule | 400 mg |
| | | | | capsule |

Study Procedures

Safety Assessments

Complete Physical Examination

A complete physical examination is performed by a medically qualified professional at Screening, on Study Day 21, and at the 30-Day Follow-up Visit. A review of all major body systems is performed, including skin, head/ears/eyes/nose/throat (HEENT), respiratory, cardiovascular, gastrointestinal, endocrine/metabolic, genitourinary, neurological, blood/lymphatic, and musculoskeletal. A fecal specimen is collected by rectal examination at Screening and on Study Day 21 and tested for the presence of occult blood. The rectal exam is included at the 30 Day Follow-Up Visit only if fecal occult blood was detected in the sample collected on Study Day 21. Assessments of height (height will be measured only at Screening visit), weight, and vital signs (systolic and diastolic blood pressure, pulse, temperature, and respirations) are included.

Brief Physical Examination

A brief physical examination will be performed by a medically qualified professional at Study Day 1. A review of body systems will be assessed, as appropriate, evaluating and documenting any changes from the previous visit. Any clinically significant changes will be followed up per standards of good medical practice. The evaluation of previous and new adverse events is to be documented. Other body systems will be assessed as appropriate. All brief physical examination data will be recorded on the appropriate CRF.

Clinical Laboratory Analyses

Blood and urine samples for clinical laboratory analyses are collected at Screening and analyzed at a central laboratory. If a value is outside the laboratory's normal range, an Investigator will indicate the deviation's significance. If significant, laboratory tests are repeated. Samples are analyzed for the parameters listed in the table below:

TABLE
Blood analyses performed

| General | Coagulation | Urinalysis | Hematology |
|---|---|---|---|
| Sodium | PT | Specific gravity | RBC |
| Potassium | PTT | pH | Hemoglobin |
| Chloride | INR | Blood | Hematocrit |

| | | |
|---|---|---|
| Bicarbonate | Protein | MCV |
| Total protein | Glucose | MCH |
| Albumin | Bilirubin | MCHC |
| Calcium | WBC | Reticulocytes |
| Magnesium | RBC | Platelets |
| Phosphorus | Epithelial cells | WBC |
| Glucose | Bacteria | Differential |

| | | |
|---|---|---|
| BUN | Casts | Bands/Stabs |
| Creatinine | Crystals | Eosinophils |
| Uric Acid | Ketones | Basophils |
| Total bilirubin | Occult Blood | Lymphocytes |

| | |
|---|---|
| Direct bilirubin | Monocytes |
| Alk phos | |
| LDH | |
| AST (SGOT) | |
| ALT (SGPT) | |
| Cholesterol | |
| LDL | |
| HDL | |
| Triglycerides | |

Electrocardiogram

A standard 12-lead resting electrocardiogram (ECG) is performed at the Screening Visit, Study Day 1, approximately 2-3 hours after administration of the first dose of study drug; Study Day 21, approximately 2-3 hours after administration of the last dose of study drug; End of Study 30-Day Follow-up Visit, or as clinically indicated during the study. A central cardiologist reviews all ECGs at the end of treatment for each cohort.

Pharmacokinetic Assessments

Blood and urine samples are collected on Study Day 21 from fasted participants for clinical chemistries, hematology, coagulation parameters, and urinalysis. Blood samples are collected on Study Day 21 at 0.5, 1, 2, 4, and 6 hours, and, if possible, 8 and 24 hours, after administration of the final dose of study drug. The total amount of blood collected for the pharmacokinetic analyses is approximately 64 mL. All blood samples for PK/PD analysis are analyzed for both R-flurbiprofen and S-flurbiprofen, and the extent of bioinversion of R-flurbiprofen to S-flurbiprofen is assessed.

Aβ38, Aβ40, Aβ42 in Plasma and in Cerebrospinal Fluid

After collection of cerebrospinal fluid (CSF) by lumbar puncture, CSF levels of Aβ40 and Aβ42 are measured by a sandwich enzyme linked immunosorbent assay (ELISA) with increased sensitivity for low levels of Aβ.

Clinical Evaluations/Measures of Cognition

The Mini-Mental State Examination (MMSE) is administered to the subject at screening. The MMSE briefly evaluates orientation, memory, attention and calculation, language (naming, comprehension, repetition, writing), and ability to copy 2 intersecting pentagons. The maximum score is 30 points, with lower scores indicating more severe cognitive impairment.

Timing of Assessments

Screening Visit

Subjects who are interested in participating will provide information needed to assess eligibility criteria at this visit. Subjects who meet eligibility criteria provide signed informed consent to study personnel. The MMSE will be administered. Subjects' medical history is reviewed, including prescribed and over-the-counter medications and history of NSAID use for the preceding 60 days. Vital signs are recorded and a complete physical and neurological examination is conducted. A fecal specimen is collected by rectal examination and tested for the presence of occult blood. An ECG is obtained and blood and urine samples collected for clinical chemistries, hematology, coagulation parameters, and urinalysis.

Baseline/Randomization Visit/Study Day 1

Study Day 1's visit, scheduled within 30 days of the Screening Visit, is scheduled for the morning; subjects fast (no fluids or food) from midnight the night before. On arrival at the clinic, subjects undergo a brief physical examination, and blood and urine samples are collected for clinical chemistries, hematology, coagulation parameters, and urinalysis. After being assigned a randomization number and treatment group, CSF is collected via lumbar puncture, and a blood sample is drawn and plasma prepared for Aβ measurements. As soon as possible after the lumbar puncture, subjects take their first dose of the study medication.

A standard 12-lead resting electrocardiogram (ECG) is performed approximately 2-3 hours after administration of the first dose of study drug, and an additional blood sample drawn 3 to 6 hours after administration of the first dose of study drug to analyze for evidence of bioinversion. Participants receive Telephone Visits on Study Days 7, 14 and 20 to verify compliance.

Study Day 21 Visit

Similar to the previous clinic visit, the subject is requested to fast overnight before coming to the clinic. Blood, urine and fecal samples are collected and analyzed as before. The subjects take their final dose of study drug at the clinic, administered by blinded study personnel, are given a standardized meal, and are questioned about AEs during the previous week. Subjects then undergo a complete physical examination. A standard 12-lead resting ECG is performed approximately 2-3 hours after administration of the final dose of study drug. Additional blood samples for PK analysis are collected on Study Day 21 at 0.5, 1, 2, 4, and 6, and if possible, 8 and 24, hours after administration of the final dose of study drug. Within 6 hours following the last dose of study drug, the subject will undergo lumbar puncture for collection of CSF. Approximately 5 to 6 patients will have lumbar punctures during each of the following intervals after the last dose: 0-2 hours, 2-4 hours, and 4-6 hours. Subjects will return to the clinic approximately 30 days after the last day of study drug administration for a complete physical examination.

The schedule of assessments is as presented in the table below:

TABLE
Schedule of Assessments

| | Study Day | | | | | | | | |
|---|---|---|---|---|---|---|---|---|---|
| | | | | | | | | | 30-Day |
| | | | | | | | | | Follow- |
| | Screen −30 to −1 | −1 | 1 | 3 | 7 | 14 | 20 | 21 | up Visit |
| Phone Visit | | X | | X | X | X | X | | |
| Clinic Visit | X | | X | | | | | X | X |
| Informed Consent | X | | | | | | | | |
| Study Drug BID¹ | | | X | X | X | X | X | X¹ | |
| Medical History | X | | | | | | | | |
| Randomization | | | X | | | | | | |
| Brief Physical Exam | | | X | | | | | | |
| Complete | X | | | | | | | X | X |
| Physical Exam | | | | | | | | | |
| MMSE | X | | | | | | | | |
| ECG | X | | X | | | | | X | X |
| Blood and urine | X | | X | | | | | X | X |
| collection for | | | | | | | | | |
| safety evaluations | | | | | | | | | |
| Blood draw for plasma Aβ | | | X | | | | | X | |
| Lumbar puncture | | | X | | | | | X | |
| Repeated blood | | | | | | | | X² | |
| draw for PK² | | | | | | | | | |
| Fecal occult | X | | | | | | | X | X³ |
| blood test³ | | | | | | | | | |
| Blood draw for | | | X | | | | | | |
| assessment of | | | | | | | | | |
| bioinversion | | | | | | | | | |
| AE Monitoring | | | X | X | X | X | X | X | X |
| Fasting overnight | | X⁴ | | | | | X⁴ | | X |
| before visit⁴ | | | | | | | | | |

¹Study drug will be taken twice daily Study Day 1 through Study Day 20. Only the morning dose of study drug will be taken on Study Day 21.
²Blood draws will occur at 0.5, 1, 2, 4, and 6 hours after last dose of Study Drug. If possible, a blood sample will be collected 8 hours and 24 hours after administration of the final dose.
³Only if the test was positive on Study Day 21.
⁴Fasting to start at 10 pm and continue fasting until after the first dose and lumbar puncture.

Statistical Considerations

Safety and tolerability endpoints include adverse events, vital signs, physical examinations, ECG, clinical laboratory tests (serum chemistry, hematology, and urinalysis, and fecal occult blood), and bioinversion of (R)-flurbiprofen to (S)-flurbiprofen. Bioinversion of R-flurbiprofen to (S)-flurbiprofen will be assessed by measurement of plasma concentrations of individuals (R)-enantiomers or (S)-enantiomers. Other endpoints are plasma pharmacokinetic parameters, CSF concentration of R-flurbiprofen, and CSF and plasma Aβ concentration, including the amyloid species Aβ42, Aβ40 and Aβ38. Pharmacokinetics are assessed by measurement of plasma concentrations of R-flurbiprofen over time. Cerebrospinal fluid levels of R-flurbiprofen over time are assessed if possible, using the actual time of the CSF measurement and combining data from all subjects. The exploratory biomarkers beta amyloid fragments Aβ38, Aβ40, and Aβ42 are measured in plasma and in CSF before and after 21 days administration of different dose levels of R-flurbiprofen. Baseline levels of biomarkers in CSF and plasma are used as covariates for analyzing treatment differences in final levels of biomarkers in CSF and plasma. Actual or estimated plasma or CSF levels of R-flurbiprofen are used as quantitative terms in predicting levels of biomarkers.

Because this is a safety study in healthy subjects, no efficacy analyses are performed. Assessment of biomarkers may provide evidence of Aβ-lowering activity that would provide a basis for association with efficacy assessments in future trials in patients with AD.

Safety Analyses

Subject incidence rates of all adverse events will be tabulated by body system, preferred term, and severity. Tables and/or narratives of “on-study” deaths, serious and significant adverse events, including early withdrawals due to adverse events, will also be provided. Statistical analysis comparing the incidence rates between groups will be performed if there are sufficient numbers of events.

Shift tables from the baseline visit to the end-of-study visit re presented for physical examination outcomes and all measured laboratory parameters. Change from baseline in quantitative laboratory parameters are compared between treatment groups. Descriptive statistics for vital signs and ECG parameters are provided for each visit and for the change from baseline. Treatment groups are compared for differences in change from baseline.

Incidence rates of bioinversion of R-flurbiprofen to (S)-flurbiprofen will be summarized by treatment group and compared between groups.

Other Analyses

The plasma concentration time profile of R-flurbiprofen is analyzed using nonlinear modeling, and preliminary estimates of PK parameters are obtained. Estimates include individual and mean half-life, clearance, AUC, Cₘₐₓ, Tₘₐₓ, and average steady-state concentration. Cerebrospinal fluid levels of R-flurbiprofen are summarized. If plasma concentrations over time are similar to those found in other patient populations, Bayesian estimation is used to estimate population PK parameters, allowing the PK parameters observed in this population to be evaluated in the context of data observed in additional clinical studies. Estimates include the mean and individual PK parameters as well as the magnitude of intersubject variability.

Summary statistics are tabulated by treatment group for the Study Day 21 plasma and CSF levels of Aβ42, Aβ40 and Aβ38. Change from baseline in levels of Aβ42 in the CSF are compared between groups using analysis of covariance with baseline CSF level of Aβ42 as a covariate. The relationship between cardioprotective aspirin usage and change in level of Aβ42 is determined.

Study Medication: R-Flurbiprofen-Packaging and Formulation

All study dosage forms (R-flurbiprofen 200-mg, 400-mg and placebo capsules) are filled into high density polyethylene bottles (HDPE) capped using child-resistant closures with induction inner seals. Bottles are packaged in kits containing 2 bottles (1 Bottle A and 1 Bottle B) that contain the doses required for 1 subject to complete Study Days 1 through 20.

Regulatory and Ethical Obligations

Study Conduct

The study is conducted in accordance with the Alzheimer's Disease Cooperative Study (ADCS) standard operating procedures. These standards respect the following guidelines:
E6 Good Clinical Practice: Consolidated Guidance (International Conference on Harmonisation of Technical Requirements for the Registration of Pharmaceuticals for Human Use [ICH], May 1996).
US Title 21 of the Code of Federal Regulations (21 CFR) dealing with clinical studies (21 CFR Parts 11, 50, 54, 56,312, and 314).
Declaration of Helsinki, concerning medical research in humans (“Recommendations Guiding Physicians in Biomedical Research Involving Human Patients,” Helsinki 1964, amended Tokyo 1975, Venice 1983, Hong Kong 1989 and revised version of Somerset West, Republic of South Africa, October, 1996).

Elements of an Informed Consent

A signed ICF, in compliance with 21 CFR Part 50, shall be obtained from each subject prior to entering the study or performing any unusual or non-routine procedure that involves a risk to the subject (see Appendix D). The informed consent document should be prepared in the language(s) of the potential patient population.

Before a subject's participation in the trial, the Investigator is responsible for obtaining written informed consent from the subject after adequate explanation of the aims, methods, anticipated benefits, and potential hazards of the study and before any protocol-specific screening procedures or any study medications are administered.

The acquisition of informed consent should be documented in the subject's medical records, as required by 21 CFR Part 312.62, and the ICF should be signed and personally dated by the subject and by the person who conducted the informed consent discussion (not necessarily an Investigator). The original signed ICF should be retained in accordance with institutional policy, and a copy of the signed consent form should be provided to the subject.

### 2.6 Example 6

### Clinical Investigation of R-Flurbiprofen for Alzheimer'S Disease

This Example provides a randomized, double-blind, placebo-controlled study of the effect of daily treatment with R-flurbiprofen on measures of cognitive and global function in subjects with mild to moderate dementia of the Alzheimer's type.

Clinical Hypothesis

Hypothesis: treatment with R-flurbiprofen at a dose that is well tolerated slows the decline in cognitive and global function of subjects with mild to moderate dementia of the Alzheimer's type as measured by standard instruments including ADAS-cog, CDR-sb, NPI, ADCS-ADL, CIBIC+, and MMSE.

Study Design

Study subjects are diagnosed as having mild to moderate dementia of the Alzheimer's type, and have a Mini Mental State Examination score (MMSE)≧15 and ≦26. Subjects may be taking acetylcholinesterase (AChE) inhibitors provided the dose has been stable for at least 3 months. Subjects will be stratified at randomization for use/non-use of AChE inhibitors. A target of 201 subjects (67 subjects per arm) in 3 treatment groups are enrolled for 12 Months with optional follow-on treatment after Month 12 (2 treatment groups).

Subject Eligibility

Subjects in this study have mild to moderate dementia of the Alzheimer's type and meet the entry criteria as follows.

Inclusion Criteria

Subjects meet all of the following inclusion criteria during screening in order to participate in the study:
Have had a diagnosis of dementia according to the DSM IV (TR) and meet the NINCDS-ADRDA criteria for probable Alzheimer's disease.
Have a CT or MRI within the past 12 months demonstrating absence of clinically significant focal intracranial lesion. If no scan is available in the previous 12 months, then a CT scan will be obtained.
Have a screening MMSE score≧15 and ≦26.
Have a screening Modified Hachinski Ischaemic score<4.
Men or women ages >55 years and living in the community at the time of enrollment (i.e., not living in a rest home or nursing care facility).
Signed the subject Informed Consent Form (ICF) and is willing and able to participate for the duration of the study.
Ability to read and understand English to ensure compliance with cognitive testing and study visit procedures.
Six years of education, or sufficient work history to exclude mental retardation.
Female subjects must be surgically sterile or postmenopausal for >1 year.
Chronic aspirin use will be limited to cardioprotective therapy (e.g., <325 mg aspirin per day) for the duration of the study.
Subjects taking AChE inhibitors may be enrolled provided their treatment dose has been stable for at least 3 months prior to screening; subjects not taking AChE inhibitors may be enrolled if treatment with these agents is contraindicated or ineffective. Subjects previously treated with AChE inhibitors must be off drug for at least 30 days prior to screening.
Subjects must have a reliable caregiver who can read, understand and speak English, accompany them to each clinic visit, and is willing to sign the caregiver Assent Form. Caregiver must either live with the subject or see them on at least 4 days per week, with contact sufficient to insure meaningful assessment of changes in subject behavior with time, and must be prepared to verify daily compliance with study medication.
Subjects taking antidepressant, antipsychotic, and/or anxiolytic drugs, vitamin E and/or Gingko biloba are eligible, provided that the dose has been stable for at least 3 months prior to randomization.
Adequate vision and hearing to participate in study assessments.

Exclusion Criteria

Subjects with any of the following exclusion criteria do not participate in the study:
Treatment with memantine for AD within 30 days prior to screening.
Current evidence or history in the past 2 years of epilepsy, focal brain lesion, head injury with loss of consciousness and/or immediate confusion after the injuries, or DSM-IV (TR) criteria for any major psychiatric disorder including psychosis, major depression, bipolar disorder, alcohol or substance abuse.
History of hypersensitivity to flurbiprofen or other NSAIDs including COX-2 specific inhibitors.
Chronic use of NSAIDs at any dose or aspirin>325 mg per day, taken on more than 7 days per month for the 2 months prior to Day 1.
History of upper GI bleeding requiring transfusion or surgery within the past 3 years.
Documented evidence of active gastric or duodenal ulcer disease within the past 3 months.
History of NSAID-associated ulcers.
History of, or evidence of, active malignancy, except for basal cell carcinoma or squamous cell carcinoma of the skin, within the 24 months prior to entry. Men with prostate cancer may be enrolled at the discretion of the Sponsor.
Chronic or acute renal, hepatic or metabolic disorder defined by:
   Creatinine>1.5 mg/dL
   AST>2.5×Upper Limit of Normal (ULN)
   ALT>2.5×ULN
Use of any investigational therapy within 90 days, or 5 half-lives, whichever is longer, prior to screening.
Major surgery and related complications not resolved within 12 weeks prior to Day 1.
Uncontrolled cardiac conditions (New York Heart Association Class III or IV, as described in Appendix B).
Anticoagulant therapy such as warfarin within 12 weeks prior to randomization.
Treatment with any CYP2C9 inhibitor within a 2 week period prior to randomization. The following drugs and herbal preparations are examples of CYP2C9 inhibitors: amiodarone, fluconazole, fluvoxamine, isoniazid, phenylbutazone, probenicid, sulfamethoxazole, sulfaphenazole, trimethoprim, zafirlukast; danshen (Salvia miltiorrhiza); Lycium barbarum.
Treatment with the CYP2C9 substrates fluvastatin, tolbutamide, or glyburide (glibenclamide).

Drug Dosage, Administration, and Schedule

The drug dosage, administration and schedule are summarized below. Subjects are instructed to take 1 tablet from Bottle A and 1 tablet from Bottle B 2 times per day. The intraday dosing interval is approximately 12 hours. Study drug should be taken at approximately the same time each day during the participation in this 12-month study. The total daily doses for the 3 arms of the study are:
800 mg R-flurbiprofen (given as one 400 mg R-flurbiprofen tablet and 1 placebo tablet, BID)
1600 mg R-flurbiprofen (given as two 400 mg R-flurbiprofen tablets, BID)
Placebo (given as two placebo tablets, BID)
   Study medication may be taken with or without food.

Concomitant Therapy

Concomitant medications are assessed at all study visits. Concomitant medications are prescribed or over-the-counter medications and should be consistent with the inclusion/exclusion criteria. The potential for drug-drug interactions exists whenever 2 or more drugs are co-administered. In particular, flurbiprofen has been shown to inhibit the metabolism of drugs that are substrates of the enzyme cytochrome P450 (CYP) 2C9.

Concomitant medications and herbal preparations should be closely monitored and recorded by the Investigator, and prohibited at his or her discretion. The possibility of interaction with the study drug cannot be ruled out, as controlled clinical trials have not been conducted. Proscribed therapy during the study period includes:
Initiation of, or change in dosage of, AChE inhibitors.
Treatment with memantine.
More than 7 days of NSAID use or aspirin>325 mg/day per month, including COX-2 specific inhibitors. (Use of cardioprotective doses of aspirin≦325 mg/day is allowed.)
CYP2C9 substrates: R-flurbiprofen has been shown to inhibit the metabolism of drugs that are substrates of the enzyme CYP2C9. Investigators will remove subjects from the study if treatment of study subjects with fluvastatin, diclofenac, and the oral hypoglycemic agents, tolbutamide and glyburide (glibenclamide) becomes necessary during the study. It is recommended that Investigators take appropriate precautionary measures if anticoagulant therapy becomes necessary, such as close monitoring of coagulation status for those subjects that require warfarin while on study.
CYP2C9 Inhibitors: Concurrent use of CYP2C9 inhibitors (e.g., amiodarone, fluconazole, fluvoxamine, isoniazid, miconazole, phenylbutazone, probenicid, sulfamethoxazole, sulfaphenazole, teniposide, trimethoprim, zafirlukast; danshen [Saliva miltiorrhiza]; Lycium barbarum).
Ansaid®, Froben® or any other flurbiprofen-containing medication.
Other investigational medication or devices.
Cytotoxic chemotherapy.

Study Procedures

Efficacy Assessments

It is preferable to administer the efficacy assessments at approximately the same time of day for each subject throughout the trial.

ADAS-cog

The Alzheimer's Disease Assessment Scale cognitive subscale (ADAS-cog) is a psychometric instrument that evaluates memory, attention, reasoning, language, orientation and praxis. The ADAS-cog is administered by a qualified professional to assess change in cognitive function. Administration takes place on Day 1, Month 3, Month 6, Month 9, Month 12 or Early Termination Prior to Month 12, Month 15, Month 18, Month 21, and Month 24 (or End of Study).

CDR-sb

The Clinical Dementia Rating-sum of boxes (CDR-sb) is a clinical scale that rates the severity of dementia as absent, questionable, mild, moderate or severe. The score is based on interviews with the subject and caregiver, using a structured interview to assess 6 domains: memory, orientation, judgment and problem-solving, community affairs, home and hobbies, and personal care. Training is conducted to standardize administration across sites. This instrument is administered by an experienced rater who also administers the CIBIC+ and who is uninvolved with other assessments of the subject. Administration takes place on Day 1, Month 3, Month 6, Month 9, Month 12 or Early Termination Prior to Month 12, Month 15, Month 18, Month 21, and Month 24 (or End of Study).

NPI

The Neuropsychiatric Inventory (NPI) is designed to evaluate a broad range of psychopathology in AD based on an interview with the caregiver by a qualified professional. Administration will take place on Day 1, Month 6, and at Month 12 or Early Termination Prior to Month 12.

ADCS-ADL

The Alzheimer's Disease Cooperative Study-Activities of Daily Living (ADCS-ADL), which is designed to assess changes in practical function in AD patients, will be administered by a qualified professional. Caregivers are queried as to whether subjects attempted each item in the inventory during the prior 4 weeks and their level of performance. This instrument includes items to assess activities from traditional scales (grooming, dressing, walking, bathing, feeding, toileting) as well as instrumental ADL scales (shopping, preparing meals, using household appliances, keeping appointments, reading). Administration will take place on Day 1, Month 6, and at Month 12 or Early Termination Prior to Month 12.

CIBIC+

blood pressure, pulse, temperature, and respirations) are included. Review of laboratory results is evaluated for changes. Clinically significant changes are followed up per standard of care practice.

Electrocardiogram

A standard 12-lead resting electrocardiogram (ECG) is performed at screening and at Month 12 or Early Termination Prior to Month 12, and Month 24 (or End of Study). It is preferred to have the Month 12 or Early Termination prior to Month 12 ECG conducted prior to venipuncture. The ECG readings and, if available, the computer analysis, will be reviewed locally by an Investigator. The ECG report is reviewed, signed, and dated by the Investigator. Patients with clinically significant ECG findings are referred for follow-up as deemed appropriate by the Investigator.

Monthly Phone Visits

Monthly phone conversations with the caregiver are conducted to assess possible AEs and compliance with study medication and visit schedules. The phone visits are conducted during Week 2, Months 2, 4, 5, 7, 8, 10, 11, 13, 14, 16, 17, 19, 20, 22, 23 (see Appendix A, Schedule of Assessments).

Vital Signs

Vital signs (systolic and diastolic blood pressure, pulse, temperature, and respirations) are assessed at each of the following visits: at Screening, Day 1, Month 1, Month 3, Month 6, Month 9, Month 12 or Early Termination Prior to Month 12, Month 15, Month 18, Month 21, Month 24 (or End of Study), and 30-Day Off-Drug Follow-up.

Hematology, Biochemistry, and Urinalysis

Blood and urine samples for clinical laboratory analyses are collected at Screening, Day 1, Month 1, Month 3, Month 6, Month 9, Month 12 or Early Termination Prior to Month 12, Month 15, Month 18, Month 21, Month 24 (or End of Study), 30-Day Off-Drug Follow-up, and analyzed to determine whether a value is outside the laboratory's normal range, and if so, whether the deviation is clinically significant. If clinically significant, laboratory tests will be repeated according to good medical practice. Approximately 17 ml of blood is collected for clinical laboratory analyses at each visit. An abnormal laboratory value will be reported as

The Clinician Interview Based Impression of Change plus caregiver input (CIBIC+) is an instrument to assess global function, based on an interview with the caregiver. The CIBIC+ will be administered by an experienced rater who also administers the CDR-sb and is uninvolved with other assessments of the subject. Administration will take place on Day 1, Months 3, 6, 9 and at Month 12 or Early Termination Prior to Month 12. The Day 1 interview will be recorded on videotape for study purposes.

MMSE

The Mini Mental State Examination (MMSE) is a frequently used screening instrument for AD studies. This instrument evaluates orientation, memory, attention, concentration, naming, repetition, comprehension, the ability to create a sentence and to copy two intersecting polygons. The MMSE is conducted during screening to establish eligibility. It is administered at Month 6, Month 12 or Early Termination Prior to Month 12, Month 18, and Month 24 (or End of Study) to evaluate change in subject assessment.

Safety Assessments

Complete Physical Examination

A complete physical examination is performed by a medically qualified professional at screening and at Month 12 or Early Termination Prior to Month 12, and Month 24 (or End of Study). A review of all major body systems, including skin, head/ears/eyes/nose/throat (HEENT), respiratory, cardiovascular, gastrointestinal, endocrine/metabolic, genitourinary (if clinically relevant), neurological, blood/lymphatic, and musculoskeletal systems, is performed. Assessments of height (height is measured only at the Screening Visit), weight, and vital signs (systolic and diastolic blood pressure, pulse, temperature, and respirations) are included. All complete physical examination data will be recorded on the appropriate source documents.

Brief Physical Examination

A brief physical examination will be performed by a medically qualified professional at Months 1, 3, 6, 9, 15, 18, 21 and 30-Day Off-Drug Follow-up. A review of body systems will be assessed as appropriate evaluating and documenting any changes from previous visit. Assessment of vital signs (systolic and diastolic an AE only if it involves therapeutic medical intervention, if the Investigator considers it to be an AE, or if it leads to study discontinuation. Should the hemoglobin value decrease 1.5 g/dL or more from the baseline value at any visit, standard of care testing for occult blood in the stool is recommended. Further follow-up guidance is found in the Suggested Algorithm for Potential Upper GI Events (see Appendix C).

Statistical Considerations

Study Design

Primary Objective:

Evaluation of the change in cognition and global function, as measured by ADAS-cog and CDR-sb, in subjects with AD treated with 1 of 2 dose levels of R-flurbiprofen.

Secondary and Exploratory Objectives

To assess changes in activities of daily living as measured by ADCS-ADL (secondary objective) and to assess changes in global performance, cognition and behavior as measured by CIBIC+, MMSE and NPI (exploratory objectives).

Additional Observations

Additional observations of this study are to evaluate:
Safety of R-flurbiprofen treatment in this subject population
Population PK of R-flurbiprofen and bioinversion of R-flurbiprofen

Follow-on Treatment Objectives

To provide an opportunity for treatment with R-flurbiprofen to subjects originally randomized to placebo in the double-blind placebo-controlled portion of the study and to permit all subjects to continue on treatment with R-flurbiprofen if they so choose; and to assess long-term changes in cognition and global function as measured by ADAS-cog, CDR-sb and MMSE and to assess long-term safety in AD patients.

Study Endpoints, Subsets, and Covariates

The primary efficacy endpoints are the rate of change in the CDR-sb and the ADAS-cog, using a model based on slopes. The secondary efficacy endpoint is the score on the ADCS-ADL. Exploratory endpoints will be CIBIC+, MMSE and NPI.

Safety endpoints include incidence of AEs, changes from baseline in physical examination and clinical laboratory test results. Additional endpoints are PK parameters based on measurements from blood samples taken throughout the study.

Efficacy analyses for primary, secondary and exploratory endpoints include the baseline score as a covariate, and also a term for the stratification variable: use or non-use of AChE inhibitor at baseline.

An Intent to Treat (ITT) approach is used, in which all randomized subjects who receive any study treatment and have a post-baseline efficacy assessment will be included in the ITT population using an appropriate imputation method. A Per Protocol analysis population will include all subjects in the ITT population who did not have any major protocol violations. Major protocol violations is determined prior to unblinding, based on observed data. All efficacy analyses are repeated for this population. Any differences in the efficacy results between the 2 analysis populations are investigated and explained.

The primary efficacy analysis is performed on the ITT population, and compares CDR-sb and ADAS-cog scores between the 800 mg BID treatment group and the placebo group. Two-sided tests for both the CDR-sb and the ADAS-cog are performed using a nominal alpha=0.05, adjusted for the interim analysis. A secondary efficacy analysis compares the 400 mg BID treatment group to the placebo group also with a two-sided test. The type I error rate is adjusted for multiple comparisons.

Sample Size Considerations

Sample size calculations are based on a comparison between 2 groups of the average decline in the ADAS-cog score for each patient at 12 months. Assuming an effect size of 60% in the 800 mg BID group, and an estimated decline of 5.5 points in the placebo group, a decline 2.2 points would be observed in 12 months. With an estimated standard deviation of 6.4, a sample size of 67 per group is required to achieve 80% power with a one-sided alpha=0.05 and a 28% drop out rate.

Although the original power calculation above is based on a one-sided test using only the ADAS-cog, the primary analysis is based on achieving significance on two-sided tests for both the ADAS-cog and the CDR-sb. A secondary power calculation was performed to calculate the joint power of detecting a difference on both the ADAS-cog and the CDR-sb, using the same assumptions as above for the ADAS-cog, and assuming a mean 12 month decline of 1.57 points with a standard deviation of 2.2 for the CDR-sb. The correlation between the change in ADAS-cog and CDR-sb was assumed to be 0.29. As the original dropout rate of 28% appears overly conservative, a 20% dropout rate was used for this secondary power calculation. Although any effect size over 30% would be considered clinically meaningful for a drug with a disease modifying effect, an effect size of 80% is reasonable based on the literature.

Using the above assumptions, this study has 80% power for detecting an 80% effect size for both the ADAS-cog and the CDR-sb using a two-sided test, and a nominal alpha=0.05. Additionally, an effect size of 80% can be detected with approximately 50% power using a two-sided test at alpha=0.01.

Planned Methods of Analysis

General Considerations

Demographic and other baseline characteristics are summarized for all subjects in both the ITT analysis population and the Per Protocol analysis population. Subject height, weight, and age is summarized and tabulated. Treatment groups are assessed statistically for similarity at baseline and results are compared between the ITT and Per Protocol analysis populations, and adjustments or subset analyses are performed if appropriate.

The medical history, stratification group (AChE inhibitor use/non-use at beginning of study), concomitant medications, and compliance to study therapy are summarized and tabulated by treatment group. Distribution of subjects across study sites are displayed by treatment group. Quantitative data is summarized by mean, standard error, median, and range. Counts and percentages will be presented for categorical data.

Efficacy Analyses

The primary efficacy outcomes, CDR-sb and ADAS-cog, are analyzed by comparing the rates of change in CDR-sb and ADAS-cog between the 800 mg BID group and the placebo group. The rate of change is assessed using a model based on slopes with the baseline score as a covariate, and with a factor for AChE inhibitor use/non-use at the beginning of the study. Interactions are tested and removed from the model if non-significant at the alpha=0.10 level. Two-sided p-values are used to test the treatment effect. The type I error rate are adjusted appropriately for the interim analysis. The 400 mg BID group is compared to both the 800 mg BID group and the placebo group as secondary analyses using the same model and adjusting for multiple comparisons.

The secondary efficacy outcome, change in score on ADCS-ADL, and exploratory efficacy outcomes, change in scores on CIBIC+, MMSE and NPI are analyzed using a general linear model with terms for treatment, baseline score and use of an AChE inhibitor at baseline. The interactions between treatment and baseline score and treatment and use of AChE inhibitor at baseline are tested and removed from the model if non-significant at the alpha=0.10 level.

Safety Analyses

All randomized subjects receiving any study treatment are included in the safety population; this is the primary safety analysis population.

Safety is assessed based on AE incidence, physical examinations, vital sign measurements, ECG measurements, clinical laboratory test results, and rates of bioinversion. The incidence of AEs is summarized by treatment group with counts and percentages. Descriptive statistics for vital sign and ECG measurements, by treatment and time (after dose) are provided. Plasma levels of R-flurbiprofen will be summarized by treatment group over time.

Study Medication—R-flurbiprofen and Placebo Tablets

Dose Form and Packaging

All study dosage forms (R-flurbiprofen 400 mg and placebo tablets) are filled into high-density polyethylene bottles capped using child-resistant closures with induction sealed inner seals. Bottles are packaged in kits containing 6 bottles in each kit. Each kit (3 Bottles “A” and 3 Bottles “B”) contain the doses required for 1 subject for a 3-month period. Each bottle in each kit is labeled with a 2-part 3-panel double-blind bottle label with detachable blinding panel that is removed immediately prior to dispensing the bottle to the subject. A new kit is dispensed to each subject every 3 months thereafter until the subject has completed 12 months of dosing. A total of 4 kits will be dispensed to each subject completing the full 12 months dosing period of the study.

Labeling

Each kit and the bottles contained therein are individually labeled with a unique and randomized kit number that exactly identify the contents of each bottle once the blinding is broken. The blinded portion of the label contains the identity of the dosage form and the manufacturer's production lot number.

Key information contained on each bottle label includes a study medication expiry date and a detachable portion of the label (removed and attached to the CRF).

### 2.7 Example 7

### Treatment of Alzheimer'S Disease with R-Flurbiprofen

The R-flurbiprofen can be administered twice daily as tablets containing 400 mg of active ingredient or as a capsule containing 400 mg of the active ingredient. A higher dose can be administered to the patient in need of such treatment which can involve the patient taking e.g., a 800 mg dose of R-flurbiprofen in the morning and a 800 mg dose of R-flurbiprofen in the evening. Typically, for the treatment of mild-to-moderate Alzheimer's disease, an individual is diagnosed by a doctor as having the disease using a suitable combination of observations. One criterion indicating a likelihood of mild-to-moderate Alzheimer's disease is a score of about 15 to about 26 on the MMSE test. Another criteria indicating mild-to-moderate Alzheimer's disease is a decline in cognitive function. R-flurbiprofen can also be administered in liquid or dosage forms. The dosages can also be divided or modified, and taken with or without food. For example, the 400 mg dose can be divided into two 200 mg tablets or capsules.

Depending on the stage of the disease, the NSAID (i.e., R-flurbiprofen) can also be administered twice daily in liquid, capsule, or tablet dosage forms where the dose has various amounts of R-flurbiprofen (i.e., 850 mg, 750 mg, 700 mg, 650 mg, 600 mg, 550 mg, 500 mg, 450 mg, 350 mg, 300 mg, 250 mg, 200 mg, 150 mg, and 100 mg). Again, the dosages can also be divided or modified, and taken with or without food. The doses can be taken during treatment with over medications for treating Alzheimer's disease or symptoms thereof. For example, the NSAID can be administered in the morning as a tablet containing 400 mg of active ingredient (i.e., R-flurbiprofen) and an acetylcholine esterase inhibitor (i.e., tacrine (Cognex®), donepezil (Aricept®), rivastigmine (Exelon®), and galantamine (Reminyl®)), and/or an NMDA antagonist (i.e., memantine). It may be desirable to lower the amount of acetylcholine esterase inhibitor (and/or NMDA antagonist) and/or NSAID to avoid adverse side effects associated with higher doses of these compounds. Alternatively, the acetylcholine esterase inhibitor (and/or NMDA antagonist) and NSAID can be co-formulated into a single dosage form, i.e., liquid, tablet, capsule, etc. Patients having mild-to-moderate Alzheimer's disease undergoing the treatment regimen of this example with R-flurbiprofen doses of about 400 mg to 800 mg can experience a lessening in decline of cognitive function (as measured by the ADAS-cog or CDR sum of boxes), plaque pathology, and/or biochemical disease marker progression.

### 2.8 Example 8

### Prevention of Alzheimer'S Disease

Prior to the onset of symptoms of Alzheimer's disease or just at the very beginning stages of the disease (e.g., a patient diagnosed with MCI), patients desiring prophylaxis against Alzheimer's disease can be treated with R-flurbiprofen. Those needing prophylaxis can be assessed by monitoring assayable disease markers, detection of genes conferring a predisposition to the disease, other risks factors such as age, diet, other disease conditions associated with Alzheimer's. The patient can also be treated with a combination of an NMDA antagonist (e.g., memantine) and R-flurbiprofen to delay or prevent the onset of Alzheimer's disease or symptoms thereof.

The patient desiring prophylaxis against Alzheimer's disease or prophylaxis of a worsening of the symptoms of Alzheimer's disease can be treated with R-flurbiprofen in an amount sufficient to delay the onset or progression of symptoms of Alzheimer's disease. For example, a patient can be treated with 800 mg of NSAID (i.e., R-flurbiprofen) twice daily. Another preventive regimen involves administering to the patient 400 mg of R-flurbiprofen twice daily. These amounts of these active ingredients can be modified to lessen side-effects and/or produce the most therapeutic benefit. For example, 200 mg of R-flurbiprofen twice daily can be administered to reduce sides-effects associated with the use of higher levels of the active ingredient. The preventive treatment can also be, e.g., treatment on alternating days with R-flurbiprofen, or alternating weeks. Other preventive treatment regimens include, but are not limited to, treatment with R-flurbiprofen for 3 weeks out of every 4 weeks, or for several months followed by no treatment for a month and then treatment for several months in an alternating on/off schedule to reduce side-effects or toxicity problems.

Patients desiring or in need of prophylaxis against Alzheimer's disease undergoing the preventive regimen of this example with R-flurbiprofen doses of about 400 mg to 800 mg can decelerate or delay the onset of Alzheimer's disease or prevent the occurrence of Alzheimer's disease. It can be advantageous to utilize a low dosage prevention regimen which involves administration of pharmaceutical doses of 200 mg R-flurbiprofen twice daily.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

### 2.9 Example 9

### Pharmacokinetic Study

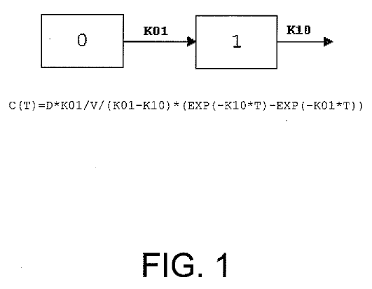

A clinical trial was conducted as described in Example 5, above. Pharmacokinetic parameters were determined using WinNonlin® Version 4.0 (Pharsight, Mountain View, Calif.). A one compartment model with first order elimination and uniform weighting was used with WinNonlin estimated parameter boundaries for absorption rate (K01), elimination rate (K10) and volume (V_F). Individual and mean value parameters derived include K10 half-life, Cₘₐₓ (maximum plasma concentration), Tₘₐₓ (time of maximum plasma concentration), AUC (area under the plasma time and concentration curve), and Cl_F (clearance). The model calculates the concentration at time T as follows:
C(T)=D*K01V/(K01−K10)*(EXP(−K10*T)−EXP(−K01*T)) (1),
where C(T) is concentration at time T; D=dose; V is volume; V_F is volume of distribution over systemic availability; K01 is the absorption rate constant; K10 is elimination rate constant; CL_F is clearance over systemic availability; and T is time. A diagram of the model is shown in FIG. 1.

Data was modeled using single dose administration (200, 400 and 800 mg for the 200 mg BID, 400 mg BID and 800 mg BID groups respectively) and expected plasma collection time points due to the fact that actual dosing and collection times were not made available at the time of analysis.

A curve stripping error occurred while modeling patients #3 and #10 in the 800 mg BID group. The error was resolved by supplying the software with the average parameter boundaries for K01, K10 and V_F, estimated by WinNonlin, from the ten other patients in the 800 mg BID group. K10_HL is the terminal half-life and as the skilled artisan recognizes, all T_{1/2} values disclosed herein are K10_HL.

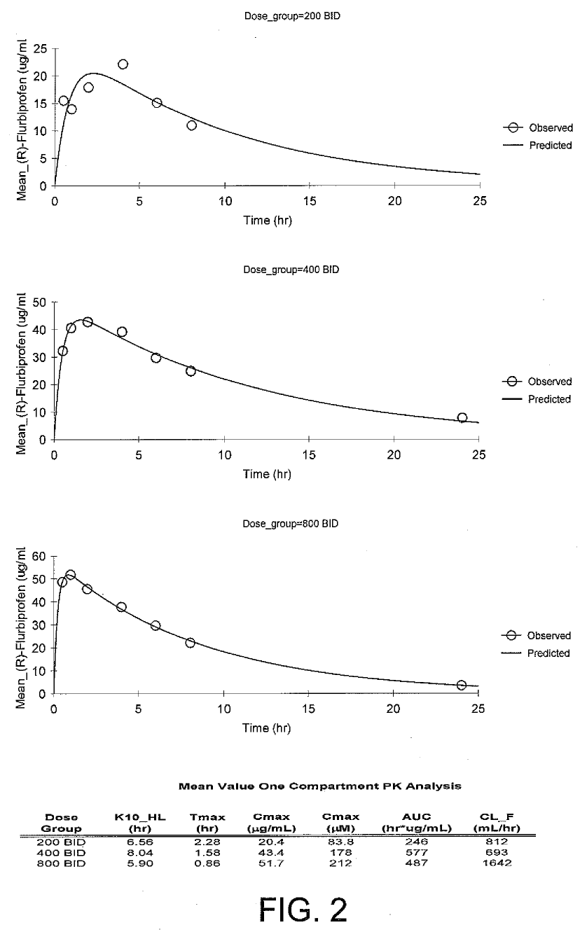

Results of the pharmacokinetic study for the 200 BID, 400 BID and 800 BID groups is shown in FIG. 2. Predicted results are shown in the table below.

Table: Predicted mean plasma concentrations for 200 b.i.d., 400 b.i.d., and 800 b.i.d. dosage by One Compartment Model.

Mean Value One Compartment PK Analysis

| Dose | K10_HL | Tmax | Cmax | Cmax | AUC | CL_F |
|---|---|---|---|---|---|---|
| Group | (hr) | (hr) | (μg/mL) | (μM) | (hr*ug/mL) | (mL/hr) |
| 200 BID | 6.56 | 2.28 | 20.4 | 83.8 | 246 | 812 |
| 400 BID | 8.04 | 1.58 | 43.4 | 178 | 577 | 693 |
| 800 BID | 5.90 | 0.86 | 51.7 | 212 | 487 | 1642 |

### 7. References Cited

All references cited herein are incorporated herein by reference in their entirety and for all purposes to the same extent as if each individual publication or patent or patent application was specifically and individually indicated to be incorporated by reference in its entirety for all purposes.

Many modifications and variations of the present invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

1-93. (canceled)

94: A unit dosage form comprising an amount of R-flurbiprofen chosen from about 350, 400, 450, 500, 550, 600, 650, 700, 750, 800, or 850 mg, and one or more pharmaceutically acceptable excipients.

95: The unit dosage form of claim 94, having about 350, 400, or 450 mg R-flurbiprofen.

96: The unit dosage form of claim 94, having about 400 mg R-flurbiprofen.

97: The unit dosage form of claim 94, which is a tablet or capsule unit dosage form.

98: A unit dosage form comprising R-flurbiprofen wherein administration of 2 unit dosage forms at steady state to a fasting patient gives a R-flurbiprofen plasma Cₘₐₓ of about 25-150 μg per mL and/or an AUC of about 250-700 hr*μg per mL.

99: The unit dosage form of claim 98, wherein said unit dosage form comprises an amount of R-flurbiprofen chosen from 350, 400, and 450 mg R-flurbiprofen.

100: The unit dosage form of claim 98, wherein said unit dosage form comprises about 400 mg R-flurbiprofen.

101: A method of treatment comprising: administering, to a person in need of treatment, about 1600 mg R-flurbiprofen per day.

102: The method of claim 101, wherein about 800 mg of R-flurbiprofen is administered twice daily.

103: The method of claim 102, wherein said administering R-flurbiprofen gives a CSF Cₘₐₓ of about 0.05 to 7.5 μg per mL.

104: The method of claim 101, wherein said person in need of treatment has mild Alzheimer's disease or mild cognitive impairment.

105: A method of treatment comprising: administering, to a person in need of treatment, an amount of R-flurbiprofen sufficient to give a plasma Cₘₐₓ of about 25-150 μg per mL at steady state and/or a plasma AUC of about 250-700 hr*μg per mL at steady state.

106: The method of claim 105, wherein said amount of R-flurbiprofen sufficient to give a plasma Cₘₐₓ of about 25-150 μg per mL at steady state and/or a plasma AUC of about 250-700 hr*μg per mL at steady state is about 800 mg R-flurbiprofen twice daily.

107: The method of claim 105, wherein said person in need of treatment has mild Alzheimer's disease or mild cognitive impairment.

108: A method of treatment comprising: administering to a patient having mild Alzheimer's disease or MCI an amount of R-flurbiprofen sufficient to lessen the rate decline of a symptom chosen from cognitive decline, biochemical disease marker progression, or plaque pathology in the patient.

109: The method of claim 108, wherein said amount of R-flurbiprofen is about 1600 mg per day.

110: The method of claim 108, wherein said amount of R-flurbiprofen gives a plasma Cₘₐₓ of about 25-150 μg per mL at steady state and/or a plasma AUC of about 250-700 hr*μg per mL at steady state.

111: A method of delaying the onset of Alzheimer's disease comprising administering to a patient an amount of R-flurbiprofen sufficient to delay or decelerate the onset of cognitive decline.

112: The method of claim 110, wherein said amount of R-flurbiprofen is about 1600 mg per day.

113: The method of claim 110, wherein said amount of R-flurbiprofen gives a plasma Cₘₐₓ of about 25-150 μg per mL at steady state and/or a plasma AUC of about 250-700 hr*μg per mL at steady state.

114: A method of treatment comprising identifying a patient having or suspected of having mild Alzheimer's disease and administering to said patient about 1600 milligrams of R-flurbiprofen per day.

115: The method of claim 114, wherein said administering comprises administering about 800 mg of R-flurbiprofen twice daily.

116: The method of claim 115, wherein said R-flurbiprofen is administered as a unit dosage form having about 400 mg R-flurbiprofen.
